(19) United States
(12) Patent Application Publication
(10) Pub. No.: US 2014/0220346 A1
(43) Pub. Date: Aug. 7, 2014

(54) MODULAR POLYMER HYDROGEL NANOPARTICLES AND METHODS OF THEIR MANUFACTURE

(71) Applicants: Memorial Sloan-Kettering Cancer Center, New York, NY (US); Massachusetts Institute of Technology, Cambridge, MA (US)

(72) Inventors: Daniel A. Heller, New York, NY (US); Jasmine Wallas, New York, NY (US); Yair Levi, Cambridge, MA (US); George W. Pratt, Waban, MA (US); Daniel G. Anderson, Sudbury, MA (US); Robert Langer, Newton, MA (US)

(73) Assignees: Memorial Sloan-Kettering Cancer Center, New York, NY (US); Massachusetts Institute of Technology, Cambridge, MA (US)

(21) Appl. No.: 14/097,212

(22) Filed: Dec. 4, 2013

Related U.S. Application Data
(60) Provisional application No. 61/733,366, filed on Dec. 4, 2012.

(51) Int. Cl. A61K 47/48; A61K 49/00

(52) U.S. Cl. CPC A61K 47/4823; A61K 49/0054; A61K 49/0073
USPC 428/402; 536/51; 536/112

(57) ABSTRACT

In certain embodiments, a nano-sized vehicle (e.g., a nanogel comprising nanoparticles) is provided herein for drug delivery with tunable biodistribution, low toxicity, and degradability, and with demonstrated targeting to bone. The composition is useful, for example, in the treatment of bone disease, particularly bone metastases from cancers such as breast, prostate, or lung cancer.

### CROSS REFERENCE

This application claims the benefit under 35 U.S.C. §119(e) of U.S. Provisional Application No. 61/733,366 filed Dec. 4, 2012.

### GOVERNMENT SUPPORT

This invention was made with government support under Grant No. RO1 DE016516 and RO1 EB000244 awarded by NIH. The government has certain rights in this invention.

### TECHNICAL FIELD

This invention relates generally to nanogels and methods of their manufacture. More particularly, in certain embodiments, nanogels for targeted tissue localization are described herein.

### BACKGROUND

Bone diseases, such as osteoporosis, metabolic diseases, and metastatic cancers, are common, but systems capable of targeting therapeutics to the bone remain limited (Wang et al., Adv. Drug Del. Rev. 2005; and Zhang et al., Chem. Soc. Rev. 2007). Nanogels—porous nanoscale hydrogel networks, are a class of nanomaterials with tunable chemical properties that facilitate targeting and delivery to specific tissues. They are intrinsically porous and can be loaded with small drugs or macromolecules by physical entrapment, covalent conjugation or controlled self-assembly (Kabanov et al., Angew Chem. Int. Edit. 2009; Naeye et al., Biomat. 2011; Raemdonck et al., Soft Mat. 2009; Zhan et al., Biomacro. 2011, 12, 3612; Oh et al., Prog. Polym. Sci 2008; Vinogradov et al., Adv. Drug Del. Rev. 2002).

The high concentration of the mineral hydroxyapatite (HA) in bone represents a target for selective delivery. Calcium ions in HA are chelated by the bisphosphonate (BP) group, which is structurally analogous to endogenous inorganic phosphate (Lawson et al., Biomed. Mater. Res. B Appl. Biomater. 2010). Systemic administration of BPs leads to deposition of these molecules on bone tissues with minimal accumulation at other sites (Deligny et al., Nucl. Med. Biol. 1990). Bisphosphonates are used to treat osteoporosis, metabolic diseases, and they may be useful for the targeting of radio-pharmaceuticals, estrogen, corticoids, anti-inflammatory agents, and proteins (Wang et al., Adv. Drug Del. Rev. 2005; Zhang et al., Chem. Soc. Rev. 2007; Wong et al., Cochrane Database Syst Rev 2002; Fleisch, Eur. Spine J. 2003; Gittens et al., Advan. Drug Deliv. Rev. 2005; Uludag et al., Biotechnol. Prog. 2000; and Uludag, Curr. Pharm. Design 2002). Polymers targeted with bisphosphonate ligand have demonstrated bone-tissue localization (Low et al., Adv. Drug Deliv. Rev. 2012).

Nanogels based on biopolymers potentially benefit from their low toxicity and biorecognitive properties. Dextran, a polysaccharide of glucose, can be recognized by C-type lectin receptors in myeloid cells and taken up by these cells (Robinson et al., Nat Immunol. 2006). Crosslinked nanogels composed primarily of dextran have been synthesized and usually contain other polymeric building blocks, such as hydroxyl ethyl methacrylate, used for free radical polymerization (Van Thienen et al., Macromol. 2005). Biodistribution of nanogels appears to be modulated in part by the attachment of surface ligands, similar to behavior of other nanoparticle types. For instance, the functionalization of poly(2-N,N-(diethylamino)ethyl methacrylate) nanogels with polyethylene glycol (PEG) results in a shift of distribution from the liver and spleen towards the lungs and kidneys (Tamura et al., Acta Biomater 2011).

Nanogels incorporating dextran have shown promise as delivery vehicles (Van Thienen et al., Macromol. 2005). However, there has been little in vivo work relating to nanogels incorporating dextran that would be applicable to their use as delivery vehicles, and no such work has been shown in bone tissue. There remains a need for vehicles for delivery of drugs and other substances to bone tissue.

### SUMMARY OF THE INVENTION

In certain embodiments, a nano-sized vehicle (e.g., a nanogel comprising nanoparticles) is provided herein for drug delivery with tunable biodistribution, low toxicity, and degradability, and with demonstrated targeting to bone. The composition is useful, for example, in the treatment of bone disease, particularly bone metastases from cancers such as breast, prostate, or lung cancer.

In one aspect, the invention is directed to a nanogel for targeted tissue localization (e.g., for preferential localization in/on bone, bone marrow, liver, and/or lymph nodes), the nanogel comprising residual (e.g., free click-able) functional groups of at least one type (e.g., unreacted groups for subsequent conjugation). In some embodiments the nanogel comprises a targeting ligand. In some embodiments, the targeting ligand is a bisphosphonate for localization in bone. In some embodiments, the residual (e.g., free click-able) functional groups are of one or more types comprising one or more of the following: alkyne, azide, thiol (sulfydryl), alkene, acrylate, oxime, maliemide, NHS (N-hydroxysuccinimide), amine (primary amine, secondary amine, tertiary amine, and/or quarternary ammonium), phenyl, benzyl, hydroxyl, carbonyl, aldehyde, carbonate, carboxylate, carboxyl, ester, methoxy, hydroperoxy, peroxy, ether, meiacetal, meiketal, acetal, ketal, orthoester, orthocarbonate ester, amide, carboxyamide, imine (primary ketimine, secondary ketamine, primary aldimine, secondary aldimine), imide, azo (diimide), cyanate (cyanate or isocyanate), nitrate, nitrile, isonitrile, nitrite (nitrosooxy group), nitro, nitroso, pyridyl, sulfide, disulfide, sulfinyl, sulfonyl, sulfino, sulfo, thiocyanate, isothiocyanate, caronothioyl, thione, thial, phosphine, phosphono, phosphate, phosphodiester, borono, boronate, bornino, borinate, halo, fluoro, chloro, bromo, and/or iodo moieties.

In some embodiments, the nanogel comprises a functionalized polymer [e.g., a polymer with at least 1%, 2%, 5%, or 7% of its monomer units (e.g., glucose subunits of a polysaccharide polymer) having attached residual functional groups (e.g., alkyne groups)]. In some embodiments, the polymer is a polysaccharide. In some embodiments, the polysaccharide is dextran.

In some embodiments, the nanogel has an average particle diameter between 5 nm and 1000 nm. In some embodiments, the average particle diameter is between 10 nm and 200 nm as measured via dynamic light scattering (DLS) of nanogel dispersed in PBS (or between 20 nm and 150 nm, or between 40 nm and 100 nm), or between 5 nm and 150 nm as measured via transmission electron micrograph (TEM) (or between 10 nm and 100 nm, or between 10 nm and 80 nm). In some embodiments, the nanogel has a substantially monodisperse particle size (e.g., has polydispersity index, Mw/Mn of less than 20, more preferably less than 10, and still more preferably less than 5, less than 2, or less than 1.5).

Certain features of other aspects of the invention are applicable to this aspect as well.

In another aspect, the invention is directed to a nanogel for targeted tissue localization (e.g., for preferential localization in/on bone, bone marrow, liver, and/or lymph nodes), the nanogel comprising a polymer and one or more ligands coupled thereto and/or therewithin, the one or more ligands comprising: (i) one or more targeting agents, (ii) one or more therapeutic agents, and/or (iii) one or more imaging agents. In some embodiments, the one or more ligands are coupled to and/or within the nanogel by at least one of (i) physical entrapment, (ii) covalent conjugation, and (iii) controlled self-assembly. In some embodiments, the nanogel further comprises residual (e.g., free click-able) functional groups of at least one type (e.g., unreacted groups for subsequent conjugation). In some embodiments, the residual (e.g., free click-able) functional groups comprise alkyne moieties and/or azide moieties. In some embodiments, the nanogel comprising one or more targeting agents comprising a bisphosphonate (e.g., for bone localization). In some embodiments, the nanogel comprises one or more targeting agents and further comprises one or more therapeutic agents selected from the group consisting of estrogen, a radio-pharmaceutical, a corticoid, an anti-inflammatory agent, and a protein. In some embodiments, the nanogel comprises one or more targeting agents and further comprises one or more imaging agents selected from the group consisting of radiolabels, radionuclides, radioisotopes, fluorophores, fluorochromes, dyes, metal lanthanides, and fluorescent proteins. In some embodiments, the one or more ligands comprises a peptide, polypeptide, and/or an antibody for binding cancer cell surface antigens/markers. In some embodiments, the one or more ligands comprises a ligand that is both a targeting agent and a therapeutic agent (e.g., bisphosphonate).

In some embodiments, the polymer is a polysaccharide. In some embodiments, the polysaccharide is dextran. In some embodiments, the nanogel has average particle diameter between 5 nm and 1000 nm. In some embodiments, the average particle diameter is between 10 nm and 200 nm as measured via dynamic light scattering (DLS) of nanogel dispersed in PBS (or between 20 nm and 150 nm, or between 40 nm and 100 nm), or between 5 nm and 150 nm as measured via transmission electron micrograph (TEM) (or between 10 nm and 100 nm, or between 10 nm and 80 nm). In some embodiments, the nanogel has substantially monodisperse particle size (e.g., has polydispersity index, Mw/Mn of less than 20, more preferably less than 10, and still more preferably less than 5, less than 2, or less than 1.5). In some embodiments, the one or more ligands are conjugated to the polymer via alkyne (alkenyl) moieties and/or azide moieties. In some embodiments, the one or more ligands are conjugated to the polymer via one or more of the following: alkyne, azide, thiol (sulfydryl), alkene, acrylate, oxime, maliemide, NHS (N-hydroxysuccinimide), amine (primary amine, secondary amine, tertiary amine, and/or quarternary ammonium), phenyl, benzyl, hydroxyl, carbonyl, aldehyde, carbonate, carboxylate, carboxyl, ester, methoxy, hydroperoxy, peroxy, ether, meiacetal, meiketal, acetal, ketal, orthoester, orthocarbonate ester, amide, carboxyamide, imine (primary ketimine, secondary ketamine, primary aldimine, secondary aldimine), imide, azo (diimide), cyanate (cyanate or isocyanate), nitrate, nitrile, isonitrile, nitrite (nitrosooxy group), nitro, nitroso, pyridyl, sulfide, disulfide, sulfinyl, sulfonyl, sulfino, sulfo, thiocyanate, isothiocyanate, caronothioyl, thione, thial, phosphine, phosphono, phosphate, phosphodiester, borono, boronate, bornino, borinate, halo, fluoro, chloro, bromo, and/or iodo moieties.

Certain features of other aspects of the invention are applicable to this aspect as well.

In another aspect, the invention is directed to a pharmaceutical composition comprising the nanogel according to any one of the preceding embodiments. In another aspect, the invention is directed to a pharmaceutical composition comprising the nanogel according to any one of the preceding embodiments and at least one of a pharmaceutically acceptable carrier, diluent, or excipient. In another aspect, the invention is directed to a composition, comprising the nanogel according to any one of the preceding embodiments, for use as a medicament. In another aspect, the invention is directed to a composition, comprising the nanogel according to any one of the preceding embodiments, for use in therapy. In another aspect, the invention is directed to a composition, comprising the nanogel according to any one of the preceding embodiments, for use in the treatment of pain. In another aspect, the invention is directed to a composition, comprising the nanogel according to any one of the preceding embodiments, for use in the treatment of one or more of: osteoarthritis, osteoporosis, bone cancer, and bone metastases. In another aspect, the invention is directed to a composition, comprising the nanogel according to any one of the preceding embodiments, for use in therapy. In another aspect, the invention is directed to a composition, comprising the nanogel according to any one of the preceding embodiments, for use in the treatment of pain associated with arthritis.

In another aspect, the invention is directed to the use of a composition, comprising the nanogel according to any one of the preceding embodiments, for the manufacture of a medicament to treat one or more of: pain, osteoarthritis, osteoporosis, bone cancer, and metastatic disease. In another aspect, the invention is directed to the nanogel according to any one of the preceding embodiments, wherein the one or more ligands comprises: (i) a small molecule drug, (ii) a peptide (e.g., a polypeptide), (iii) antibody, and/or (iv) a protein, at a concentration on the surface of the nanogel particles that enhances receptor binding affinity of the one or more ligands (e.g., to cancer cell surface antigens/markers).

In another aspect, the invention is directed to a method of manufacturing a nanogel for targeted tissue localization (e.g., a nanogel according to any one of the preceding embodiments), the method comprising: providing a first quantity of biopolymer modified with a first moiety (e.g., clickable alkyne groups); providing a second quantity of biopolymer modified with a second moiety (e.g., clickable azide groups); and crosslinking the first quantity of biopolymer and the second quantity of biopolymer in an inverse emulsion, thereby producing nanoparticles having an excess of free unreacted moieties (e.g., alkyne groups and/or azide groups) for subsequent conjugation.

In some embodiments, the first quantity of biopolymer is dextran modified with alkyne groups (e.g., with alkyne ligand substitution ratio of between 5% and 20%, or between 7% and 15% per glucose subunit) and the second quantity of biopolymer is dextran modified with azide groups (e.g., with azide ligand substitution ratio of between 2% and 10%, or between 3% and 7% per glucose subunit). In some embodiments, the first quantity of biopolymer and the second quantity of biopolymer are crosslinked in an approximately 5:1 to 1:5 ratio (e.g., 3:1 to 1:3). In some embodiments, the nanoparticles have an alkyne ligand substitution ratio of at least 1%, 2%, 5%, or 7% with respect to the total number of glucose subunits (e.g., at least 1%, 2%, 5%, or 7% of the glucose subunits have attached residual alkyne groups). In some embodiments, the first quantity of biopolymer and/or the second quantity of biopolymer has molecular weight between about 5,000 and 20,000 Da (e.g., about 10,000 Da). In some embodiments, the resulting nanogel has an average particle diameter between 5 nm and 1000 nm. In some embodiments, the average particle diameter is between 10 nm and 200 nm as measured via dynamic light scattering (DLS) of nanogel dispersed in PBS (or between 20 nm and 150 nm, or between 40 nm and 100 nm), or between 5 nm and 150 nm as measured via transmission electron micrograph (TEM) (or between 10 nm and 100 nm, or between 10 nm and 80 nm). In some embodiments, the nanogel has a substantially monodisperse particle size (e.g., has polydispersity index, Mw/Mn of less than 20, more preferably less than 10, and still more preferably less than 5, less than 2, or less than 1.5). In some embodiments, the manufactured nanogel comprises residual (e.g., free click-able) functional groups [e.g., the polymer of the nanogel has at least 1%, 2%, 5%, or 7% of its monomer units (e.g., glucose subunits of a polysaccharide polymer) having attached residual functional groups (e.g., alkyne groups)].

In another aspect, the invention is directed to at least one of (i) a therapeutic procedure, (ii) a drug delivery technique, and (iii) an imaging procedure, utilizing the nanogel of any one of the embodiments described above (e.g., with or without subsequent conjugation).

In some embodiments (of any aspect of the invention), use of the nanogel or nanogel composition comprises administration of the nanogel or nanogel composition to a subject. In some embodiments (of any aspect of the invention), the nanogel or nanogel composition is biocompatible. In some embodiments (of any aspect of the invention), the nanogel or nanogel composition is biodegradable. In some embodiments (of any aspect of the invention), the nanogel or nanogel composition is hydrolytically biodegradable. In some embodiments (of any aspect of the invention), use of the nanogel or nanogel composition is performed under physiological conditions (e.g., in vivo).

Other features, objects, and advantages of the present invention are apparent in the figures, definitions, detailed description, and claims that follow. It should be understood, however, that the figures, definitions, detailed description, and claims, while indicating embodiments of the present invention, are given by way of illustration only, not limitation. Various changes and modifications within the scope of the invention will become apparent to those skilled in the art.

### DESCRIPTION OF THE DRAWING

The Figures described below, that together make up the Drawing, are for illustration purposes only, not for limitation.

FIG. 1 depicts the assembly of nanogels and their chemical characterization. FIG. 1A depicts dextran polymer precursors with alkyne or azide ligands for click chemistry. FIG. 1B depicts the synthesis scheme for alkyne-heavy nanogels: alkyne and azide-functionalized dextrans react within an inverse emulsion. FIG. 1C depicts DLS measurements of alkyne-heavy (♦), bisphosphonate-functionalized (▪), and azide-heavy (▴) nanogels in PBS. FIGS. 1D and 1E depict transmission electron micrographs of alkyne-heavy nanogels. FIGS. 1F and 1G depict atomic force micrographs of surface-bound alkyne-heavy nanogels. Microscopy was conducted in dry conditions.

FIG. 2 depicts nanogel functionalization. FIG. 2A depicts dextranase degradation of nanogels at pH 6.0. Nanogel size increases approximately 500%. All error bars equal one standard deviation. FIG. 2B depicts fluorescently-labeled alkyne-heavy nanogel and FIG. 2C depicts bisphosphonate-derivatized nanogel binding study to surface-bound hydroxyapatite particles. FIG. 2D depicts bright-field images of hydroxyapatite particles. FIG. 2E depicts fluorescence of nanogels (F_{N}) measured relative to hydroxyapatite particle area (A_{HA}).

FIG. 3 depicts that alkyne-heavy nanogels exhibit enhanced uptake by RAW 264.7 macrophages compared to HeLa and HepG2 cells. FIG. 3A depicts quantitated total fluorescence in three cell types. FIG. 3B depicts images of fluorophore-labeled nanogels taken with a high-throughput confocal fluorescence microscopy system. Asterisks signify that means are significantly different (P<0.0001).

FIG. 4 depicts biodistribution of alkyne-heavy nanogels and bisphosphonate-functionalized nanogels (Bis-Nanogels) in SKH-1 hairless mice. FIG. 4A depicts in vivo fluorescence at 24 hours. Liver and lymph node localization is evident in the ventral view, while the dorsal view shows increased relative spinal distribution of bis-nanogels. (All mice shown in FIG. 7, N=4.) FIG. 4B depicts relative fluorescence intensity in organs ex vivo, after 24 hours. Asterisks signify that means are significantly different (P<0.05). FIG. 4C depicts FACS analysis of F4/80⁺ and co-positive F4/80⁺ Alexa-647⁺ cells in single-cell suspensions prepared from PBS- or nanoparticle-(nanogels or bis-nanogels) treated bone marrow (spinal or femural) from SHK-1 mice. Cell numbers shown in each quadrant are expressed as a percentage of the total cell population. In correlation with in vitro data (FIG. 3), bisphosphonate modification decreased F4/80/Alexa-647 co-positive cell populations in spine and femur bone marrow in vivo by 46.2% and 45.5%, respectively, as compared to Dex-treated samples (n=2 per treatment group). FIG. 4D depicts confocal images of cryosectioned femur and spinal vertebrae treated with either PBS, nanogels, or bis-nanogels, and isolated from SHK-1 mice at 24 hr. Representative images taken with a 10× objective lens are shown for both femurs and spine. Nanogel signal (Alexa-647) is shown in red. Bone marrow and bone morphological features are shown in blue. White arrows highlight the bis-nanogel-bound bone tissue. FIG. 4E depicts cryosections cortical and trabecular femoral bone tissue are shown after treatment with either nanogels or bis-nanogels. White arrows highlight bis-nanogel bound bone. FIG. 4F depicts Calcein-stained femur shows areas of co-localization of bis-nanogel fluorescence and higher calcium concentrations in the tissue.

FIG. 5 depicts nanogels as described in this application. A new class of nanogel demonstrates modular biodistribution and affinity for bone. Nanogels, 67 nm in diameter and synthesized via an astoichiometric click-chemistry-in-emulsion method, controllably display residual, free click-able functional groups. Functionalization with a bisphosphonate ligand results in significant binding to bone on the inner walls of marrow cavities, liver avoidance, and anti-osteoporotic effects.

FIG. 6 depicts ¹H NMR spectra of functionalized dextran polymer and nanogels. FIG. 6A depicts the ¹H NMR spectra of alkyne-dextran. FIG. 6B depicts the ¹H NMR spectra of azide-dextran. FIG. 6C depicts the ¹H NMR spectra of alkyne-heavy nanogels. FIG. 6D depicts the ¹H NMR spectra of azide-heavy nanogels.

FIG. 7 depicts biodistribution of alkyne-heavy nanogels and bisphosphonate-functionalized nanogels (Bis-Nanogels) in SKH-1 hairless mice at 24 hours. (Full images corresponding to FIG. 4A.) Liver and lymph node localization is evident in the ventral view (top), while the dorsal view (bottom) shows increased relative spinal distribution of bis-nanogels. A control mouse appears on the right in each image.

FIG. 8 depicts biodistribution of fluorophore-labeled azide-dextran uncrosslinked polymer 24 hours after tail vein injection. In the ventral view (left), the dextran appears to localize primarily in the lymph nodes, spleen, and peritoneal cavity. The dorsal view (right) shows some likely localization in the spine. A control mouse is on the right in each image.

FIG. 9 depicts alkyne-heavy nanogels in SHK-1 mice imaged at 24 hours (left) and 5 days (right) after tail vein injection demonstrate clearance from the body. A control mouse is on the right in both images.

FIG. 10 depicts fluorescence image of alkyne-heavy (top row) and bisphosphonate-functionalized (bottom row) dextran nanogels in organs harvested 24 hours after i.v. injection. Organs from the control animals (injected with PBS only) are on the right. (Below) Legend of organ locations.

FIG. 11 depicts relative nanogel and bis-nanogel signal found in perfused murine spinal and femur marrow supernatant, after 24 hours. P(* denotes P<0.05, *** denotes P<0.001)

FIG. 12 depicts confocal images of cryosectioned femur treated with either PBS, nanogels, or bis-nanogels, and isolated from SHK-1 mice at 24 hr. Nanogel signal (Alexa-647) is shown in grayscale on the left column and in red on the two columns on the right. Bone marrow and bone morphological features are shown using the autofluorescence of features under UV excitation in grayscale in the second and fourth columns, as well as in blue in the third column.

FIG. 13 depicts confocal images of cryosectioned spinal vertebrae treated with either PBS, nanogels, or bis-nanogels, and isolated from SHK-1 mice at 24 hr. Nanogel signal (Alexa-647) is shown in grayscale on the left column and in red on the two columns on the right. Bone marrow and bone morphological features are shown using the autofluorescence of features under UV excitation in grayscale in the second and fourth columns, as well as in blue in the third column.

FIG. 14 depicts selected, magnified regions of the confocal images from FIG. 11 corresponding to the representative images in FIG. 4D. Nanogel signal (Alexa-647) is shown in grayscale on the left. Bone marrow and bone morphological features are shown using the autofluorescence of features under UV excitation in grayscale on the right.

FIG. 15 depicts Selected, magnified regions of the confocal images from FIG. 11 corresponding to the representative images in FIG. 4D. Nanogel signal (Alexa-647) is shown in red. Bone marrow and bone morphological features are shown in using the autofluorescence of features under UV excitation in grayscale.

FIG. 16 depicts confocal images of cryosectioned cortical femur tissue treated with either PBS, nanogels, or bis-nanogels, and isolated from SHK-1 mice at 24 hr. Nanogel signal (Alexa-647) is shown in grayscale on the left column and in red on the two columns on the right. Bone marrow and bone morphological features are shown using the autofluorescence of features under UV excitation in grayscale in the second and fourth columns, as well as in blue in the third column. The bottom set of images is a selected, magnified region of the top set of images.

FIG. 17 depicts confocal images of cryosectioned trabecular femur tissue treated with either PBS, nanogels, or bis-nanogels, and isolated from SHK-1 mice at 24 hr. Nanogel signal (Alexa-647) is shown in grayscale on the left column and in red on the two columns on the right. Bone marrow and bone morphological features are shown using the autofluorescence of features under UV excitation in grayscale in the second and fourth columns, as well as in blue in the third column. The bottom set of images is a selected, magnified region of the top set of images.

FIG. 18 depicts confocal images of cryosectioned femur tissue with bis-nanogels, and isolated from SHK-1 mice at 24 hr. Tissue was treated with calcein Nanogel signal (Alexa-647) is shown in grayscale on the left column and in red on the two columns on the right. Bone marrow and bone morphological features are shown using the autofluorescence of features under UV excitation in grayscale in the second and fourth columns, as well as in blue in the third column. The bottom set of images is a selected, magnified region of the top set of images.

FIG. 19 depicts the scheme of the two-step procedure for the radiolabeling of dextran nanoparticles.

FIG. 20 depicts the synthetic route to creating DBCO-NOTA.

Table S1 depicts the results of MTS Assays after nanogel uptake by different cell types. The table shows 1050 values after 48 h incubation (MTS assay). The signal is the mean of three confluent wells. A value >1.8 signifies that no toxicity was apparent at the highest concentration used.

### DEFINITIONS

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration”: The term “administration” refers to introducing a substance into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments. In some embodiments, administration is oral. Additionally or alternatively, in some embodiments, administration is parenteral. In some embodiments, administration is intravenous.

“Agent”: The term “agent” refers to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present invention include small molecules, antibodies, antibody fragments, aptamers, siRNAs, shRNAs, DNA/RNA hybrids, antisense oligonucleotides, ribozymes, peptides, peptide mimetics, peptide nucleic acids, small molecules, etc. In some embodiments, an agent is or comprises a polymer. In some embodiments, an agent contains at least one polymeric moiety.

“Amino Acid”: As used herein, the term “amino acid,” in its broadest sense, refers to any compound and/or substance that can be incorporated into a polypeptide chain. In some embodiments, an amino acid has the general structure H2N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

“Antibody polypeptide”: As used herein, the terms “antibody polypeptide” or “antibody”, or “antigen-binding fragment thereof”, which may be used interchangeably, refer to polypeptide(s) capable of binding to an epitope. In some embodiments, an antibody polypeptide is a full-length antibody, and in some embodiments, is less than full length but includes at least one binding site (comprising at least one, and preferably at least two sequences with structure of antibody “variable regions”). In some embodiments, the term “antibody polypeptide” encompasses any protein having a binding domain which is homologous or largely homologous to an immunoglobulin-binding domain. In particular embodiments, “antibody polypeptides” encompasses polypeptides having a binding domain that shows at least 99% identity with an immunoglobulin binding domain. In some embodiments, “antibody polypeptide” is any protein having a binding domain that shows at least 70%, 80%, 85%, 90%, or 95% identity with an immunoglobulin binding domain, for example a reference immunoglobulin binding domain. An included “antibody polypeptide” may have an amino acid sequence identical to that of an antibody that is found in a natural source. Antibody polypeptides in accordance with the present invention may be prepared by any available means including, for example, isolation from a natural source or antibody library, recombinant production in or with a host system, chemical synthesis, etc., or combinations thereof. An antibody polypeptide may be monoclonal or polyclonal. An antibody polypeptide may be a member of any immunoglobulin class, including any of the human classes: IgG, IgM, IgA, IgD, and IgE. In certain embodiments, an antibody may be a member of the IgG immunoglobulin class. As used herein, the terms “antibody polypeptide” or “characteristic portion of an antibody” are used interchangeably and refer to any derivative of an antibody that possesses the ability to bind to an epitope of interest. In certain embodiments, the “antibody polypeptide” is an antibody fragment that retains at least a significant portion of the full-length antibody's specific binding ability. Examples of antibody fragments include, but are not limited to, Fab, Fab′, F(ab′)2, scFv, Fv, dsFv diabody, and Fd fragments. Alternatively or additionally, an antibody fragment may comprise multiple chains that are linked together, for example, by disulfide linkages. In some embodiments, an antibody polypeptide may be a human antibody. In some embodiments, the antibody polypeptides may be a humanized. Humanized antibody polypeptides include may be chimeric immunoglobulins, immunoglobulin chains or antibody polypeptides (such as Fv, Fab, Fab′, F(ab′)2 or other antigen-binding subsequences of antibodies) that contain minimal sequence derived from non-human immunoglobulin. In general, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity.

“Antigen”: As used herein, the term “antigen” is a molecule or entity to which an antibody binds. In some embodiments, an antigen is or comprises a polypeptide or portion thereof. In some embodiments, an antigen is a portion of an infectious agent that is recognized by antibodies. In some embodiments, an antigen is an agent that elicits an immune response; and/or (ii) an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism; alternatively or additionally, in some embodiments, an antigen elicits a cellular response (e.g., involving T-cells whose receptors specifically interact with the antigen) in an organism. It will be appreciated by those skilled in the art that a particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor, and may or may not induce a particular physiological response in an organism. In some embodiments, for example, an antigen may bind to an antibody and/or to a T cell receptor in vitro, whether or not such an interaction occurs in vivo. In general, an antigen may be or include any chemical entity such as, for example, a small molecule, a nucleic acid, a polypeptide, a carbohydrate, a lipid, a polymer [in some embodiments other than a biologic polymer (e.g., other than a nucleic acid or amino acid polymer)] etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is or comprises a recombinant antigen.

“Associated”: As used herein, the term “associated” typically refers to two or more entities in physical proximity with one another, either directly or indirectly (e.g., via one or more additional entities that serve as a linking agent), to form a structure that is sufficiently stable so that the entities remain in physical proximity under relevant conditions, e.g., physiological conditions. In some embodiments, associated moieties are covalently linked to one another. In some embodiments, associated entities are non-covalently linked. In some embodiments, associated entities are linked to one another by specific non-covalent interactions (i.e., by interactions between interacting ligands that discriminate between their interaction partner and other entities present in the context of use, such as, for example. streptavidin/avidin interactions, antibody/antigen interactions, etc.). Alternatively or additionally, a sufficient number of weaker non-covalent interactions can provide sufficient stability for moieties to remain associated. Exemplary non-covalent interactions include, but are not limited to, electrostatic interactions, hydrogen bonding, affinity, metal coordination, physical adsorption, host-guest interactions, hydrophobic interactions, pi stacking interactions, van der Waals interactions, magnetic interactions, electrostatic interactions, dipole-dipole interactions, etc.

As used herein, for example, within the claims, the term “ligand” encompasses moieties that are associated with another entity, such as a nanogel polymer, for example. Thus, a ligand of a nanogel polymer can be chemically bound to, physically attached to, or physically entrapped within, the nanogel polymer, for example.

“Biocompatible”: The term “biocompatible”, as used herein is intended to describe materials that do not elicit a substantial detrimental response in vivo. In certain embodiments, the materials are “biocompatible” if they are not toxic to cells. In certain embodiments, materials are “biocompatible” if their addition to cells in vitro results in less than or equal to 20% cell death, and/or their administration in vivo does not induce inflammation or other such adverse effects. In certain embodiments, materials are biodegradable.

“Biodegradable”: As used herein, “biodegradable” materials are those that, when introduced into cells, are broken down by cellular machinery (e.g., enzymatic degradation) or by hydrolysis into components that cells can either reuse or dispose of without significant toxic effects on the cells. In certain embodiments, components generated by breakdown of a biodegradable material do not induce inflammation and/or other adverse effects in vivo. In some embodiments, biodegradable materials are enzymatically broken down. Alternatively or additionally, in some embodiments, biodegradable materials are broken down by hydrolysis. In some embodiments, biodegradable polymeric materials break down into their component polymers. In some embodiments, breakdown of biodegradable materials (including, for example, biodegradable polymeric materials) includes hydrolysis of ester bonds. In some embodiments, breakdown of materials (including, for example, biodegradable polymeric materials) includes cleavage of urethane linkages.

“Combination Therapy”: As used herein, the term “combination therapy”, refers to those situations in which two or more different pharmaceutical agents for the treatment of disease are administered in overlapping regimens so that the subject is simultaneously exposed to at least two agents. In some embodiments, the different agents are administered simultaneously. In some embodiments, the administration of one agent overlaps the administration of at least one other agent. In some embodiments, the different agents are administered sequentially such that the agents have simultaneous biologically activity with in a subject.

“Imaging Agent”: The term “imaging agent” as used herein refers to any element, molecule, functional group, compound, fragments thereof or moiety that facilitates detection of an agent (e.g., an antibody) to which it is joined. Examples of imaging agents include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.

“Hydrolytically degradable”: As used herein, “hydrolytically degradable” materials are those that degrade by hydrolytic cleavage. In some embodiments, hydrolytically degradable materials degrade in water. In some embodiments, hydrolytically degradable materials degrade in water in the absence of any other agents or materials. In some embodiments, hydrolytically degradable materials degrade completely by hydrolytic cleavage, e.g., in water. By contrast, the term “non-hydrolytically degradable” typically refers to materials that do not fully degrade by hydrolytic cleavage and/or in the presence of water (e.g., in the sole presence of water).

“Peptide”: The term “peptide” refers to two or more amino acids joined to each other by peptide bonds or modified peptide bonds. In particular embodiments, “peptide” refers to a polypeptide having a length of less than about 100 amino acids, less than about 50 amino acids, less than 20 amino acids, or less than 10 amino acids.

“Pharmaceutically acceptable”: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

“Pharmaceutical composition”: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

“Physiological conditions”: The phrase “physiological conditions”, as used herein, relates to the range of chemical (e.g., pH, ionic strength) and biochemical (e.g., enzyme concentrations) conditions likely to be encountered in the intracellular and extracellular fluids of tissues. For most tissues, the physiological pH ranges from about 7.0 to 7.4.

“Polypeptide”: The term “polypeptide” as used herein, refers to a string of at least three amino acids linked together by peptide bonds. In some embodiments, a polypeptide comprises naturally-occurring amino acids; alternatively or additionally, in some embodiments, a polypeptide comprises one or more non-natural amino acids (i.e., compounds that do not occur in nature but that can be incorporated into a polypeptide chain; see, for example, http://www.cco.caltech.edu/~dadgrp/Unnatstruct.gif, which displays structures of non-natural amino acids that have been successfully incorporated into functional ion channels) and/or amino acid analogs as are known in the art may alternatively be employed). In some embodiments, one or more of the amino acids in a protein may be modified, for example, by the addition of a chemical entity such as a carbohydrate group, a phosphate group, a farnesyl group, an isofarnesyl group, a fatty acid group, a linker for conjugation, functionalization, or other modification, etc.

“Polysaccharide”: The term “polysaccharide” refers to a polymer of sugars. Typically, a polysaccharide comprises at least three sugars. In some embodiments, a polypeptide comprises natural sugars (e.g., glucose, fructose, galactose, mannose, arabinose, ribose, and xylose); alternatively or additionally, in some embodiments, a polypeptide comprises one or more non-natural amino acids (e.g, modified sugars such as 2′-fluororibose, 2′-deoxyribose, and hexose).

“Protein”: As used herein, the term “protein” refers to a polypeptide (i.e., a string of at least 3-5 amino acids linked to one another by peptide bonds). Proteins may include moieties other than amino acids (e.g., may be glycoproteins, proteoglycans, etc.) and/or may be otherwise processed or modified. In some embodiments “protein” can be a complete polypeptide as produced by and/or active in a cell (with or without a signal sequence); in some embodiments, a “protein” is or comprises a characteristic portion such as a polypeptide as produced by and/or active in a cell. In some embodiments, a protein includes more than one polypeptide chain. For example, polypeptide chains may be linked by one or more disulfide bonds or associated by other means. In some embodiments, proteins or polypeptides as described herein may contain L-amino acids, D-amino acids, or both, and/or may contain any of a variety of amino acid modifications or analogs known in the art. Useful modifications include, e.g., terminal acetylation, amidation, methylation, etc. In some embodiments, proteins or polypeptides may comprise natural amino acids, non-natural amino acids, synthetic amino acids, and/or combinations thereof. In some embodiments, proteins are or comprise antibodies, antibody polypeptides, antibody fragments, biologically active portions thereof, and/or characteristic portions thereof.

“Substantially”: As used herein, the term “substantially”, and grammatic equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

“Subject”: As used herein, the term “subject” includes humans and mammals (e.g., mice, rats, pigs, cats, dogs, and horses). In many embodiments, subjects are be mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

“Therapeutic agent”: As used herein, the phrase “therapeutic agent” refers to any agent that has a therapeutic effect and/or elicits a desired biological and/or pharmacological effect, when administered to a subject.

“Treatment”: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Figures are presented herein for illustration purposes only, not for limitation.

### DETAILED DESCRIPTION

It is contemplated that compositions, systems, devices, methods, and processes of the claimed invention encompass variations and adaptations developed using information from the embodiments described herein. Adaptation and/or modification of the compositions, systems, devices, methods, and processes described herein may be performed by those of ordinary skill in the relevant art.

Throughout the description, where compositions, articles, and devices are described as having, including, or comprising specific components, or where processes and methods are described as having, including, or comprising specific steps, it is contemplated that, additionally, there are compositions, articles, and devices of the present invention that consist essentially of, or consist of, the recited components, and that there are processes and methods according to the present invention that consist essentially of, or consist of, the recited processing steps.

Similarly, where compositions, articles, and devices are described as having, including, or comprising specific compounds and/or materials, it is contemplated that, additionally, there are compositions, articles, and devices of the present invention that consist essentially of, or consist of, the recited compounds and/or materials.

It should be understood that the order of steps or order for performing certain action is immaterial so long as the invention remains operable. Moreover, two or more steps or actions may be conducted simultaneously.

The mention herein of any publication, is not an admission that the publication serves as prior art with respect to any of the claims presented herein. Headers are provided for organizational purposes and are not meant to be limiting.

In certain embodiments, a new class of nanogel with controllable surface functionalization is presented herein for targeting bone, demonstrating modular biodistribution and affinity for the marrow-bone interface. In specific experimental examples, nanogels, 67 nm in diameter and composed of dextran, were synthesized via an astoichiometric click-chemistry-in-emulsion method to controllably display residual, free click-able functional groups. Following intravenous injection in mice, nanogels localized in cervical lymph nodes, liver, and the bone marrow cavities, observed in the spine and femur. Functionalization of nanogels with a bisphosphonate ligand modulated this localization, reducing liver uptake by 43% and effecting localization on the marrow-bone interface. The targeting ligand resulted in significant nanogel binding to hydroxapatite (HA) molecules on the inner walls of the marrow cavity in both cortical and trabecular bone and reduced nanogel uptake into bone marrow F4/80-positive cells. Targeted nanogels also depleted F4/80-positive cells within bone marrow, suggesting anti-osteoporotic effects.

In some embodiments, nanogel particles may be modified to change the following: size, degradation rate, drug release rate, basic polymer composition, porosity, pore size, ratio of alkyne:azide groups, total number of alkyne and azide groups, and/or the addition of a different click chemistry moiety including but not limited to thiols, alkene, acrylate, oxime, maliemide, NHS, amine, and others as described elsewhere in the application.

In some embodiments, nanogels are manufactured for targeted tissue localization. In some embodiments, methods of manufacturing nanogels comprises providing a first quantity of biopolymer modified with a first moiety (for example, clickable alkyne groups); providing a second quantity of biopolymer modified with a second moiety (for example, clickable azide groups); and crosslinking the first quantity of biopolymer in an inverse emulsion, thereby producing nanoparticles with an excess of free unreacted moieties (for example, alkyne groups and/or azide groups) for subsequent conjugation.

### Functional Groups

Nanogels are conjugated to one or more ligands through the use of one or more types of functional groups. In some embodiments, the residual (e.g., free click-able) functional groups are of one or more types comprising one or more of the following: alkyne, azide, thiol (sulfydryl), alkene, acrylate, oxime, maliemide, NHS (N-hydroxysuccinimide), amine (primary amine, secondary amine, tertiary amine, and/or quarternary ammonium), phenyl, benzyl, hydroxyl, carbonyl, aldehyde, carbonate, carboxylate, carboxyl, ester, methoxy, hydroperoxy, peroxy, ether, meiacetal, meiketal, acetal, ketal, orthoester, orthocarbonate ester, amide, carboxyamide, imine (primary ketimine, secondary ketamine, primary aldimine, secondary aldimine), imide, azo (diimide), cyanate (cyanate or isocyanate), nitrate, nitrile, isonitrile, nitrite (nitrosooxy group), nitro, nitroso, pyridyl, sulfide, disulfide, sulfinyl, sulfonyl, sulfino, sulfo, thiocyanate, isothiocyanate, caronothioyl, thione, thial, phosphine, phosphono, phosphate, phosphodiester, borono, boronate, bornino, borinate, halo, fluoro, chloro, bromo, and/or iodo moieties.

Most applications involve the covalent attachment of one or more ligands to the click-chemistry groups on the interior/exterior of the particle (the alkyne and the azide moieties). In some embodiments, covalent attachment of ligands comprises: imaging agents for PET, MRI, CT, SPECT; radionuclides for radiotherapy; sensor moieties; and drugs for the treatment of diseases, pain management/palliative therapy.

### Targeting Agents

Nanogels can be targeted to localize in preferential tissues. In some embodiments, nanogels comprise a polymer and one or more ligands coupled thereto and/or therewithin, the one or more ligands comprising: (i) one or more targeting agents, (ii) one or more therapeutic agents, and/or (iii) one or more imaging agents. In some embodiments, the nanogels comprise a targeting agent for localization within preferred tissues. In some embodiments, the nanogel comprises a targeting agent for preferred localization in/on bone, bone marrow, liver, and/or lymph nodes. In some embodiments, the nanogel comprises one or more targeting agents comprising peptides, polypeptides, proteins, antibodies, aptamers, lipids, nucleic acids, and small molecules. In some embodiments, the nanogel comprises one or more targeting agents comprising a bisphosphonate. In some embodiments, the nanogel comprises one or more ligands wherein the ligand is both a targeting agent and therapeutic agent. In some embodiments, the nanogel comprises a ligand that is both a targeting agent and therapeutic agent comprising a bisphosphonate.

In some embodiments, nanogels are used for targeted imaging and/or therapeutic applications via attachment of a ligand such as a small molecule, peptide, protein, antibody, aptamer, lipid or nucleic acid. The ligand may bind extracellularly and/or promote internalization of the particle into cells. In some embodiments, nanogels comprise one or more targeting agents comprising bisphosphonate ligands for bone localization; and peptides or antibodies for binding specific cancer cell surface antigens/markers.

### Therapeutic Agents

In some embodiments, nanogels comprises one or more ligands comprising one or more therapeutic agents. In some embodiments, nanogels comprises one or more therapeutic agents comprising hormones, enzymes, radio-pharmaceuticals, corticoids, anti-inflammatory agents, antibiotics, antivirals, antifungals, chemotherapeutics, antibodies, polypeptides, proteins, nucleic acids, aptamers, and lipids. In some embodiments, nanogels comprises a particle designed to bring about a therapeutic effect by the attachment of ligands to the surface. Due to the multivalency of nanogel particle, large binding affinities to cell surface receptors and intracellular proteins results in responses including, but not limited to the recruitment of immune responses. In some embodiments, nanogels are used in a combination therapy for a disease or condition. In some embodiments, nanogels are used in combination with treatments comprising antibodies, small molecule drugs, radiation, pharmacotherapy, chemotherapy, cryotherapy, thermotherapy, electrotherapy, phototherapy, ultrasonic therapy and surgery.

### Medical Conditions

In some embodiments, nanogels are used therapeutically to treat a disease, disorder or condition. In some embodiments, nanogels are used to pain. In some embodiments, nanogels are used to treat pain associated with arthritis. In some embodiments, nanogels are used in the treatment of bone diseases and disorders. In some embodiments, nanogels are used to treat osteoarthritis, osteoporosis, bone cancer, and bone metastases. In some embodiments, nanogels are used to treat bone disorders comprising avascular necrosis (or Osteonecrosis), bone spur (Osteophytes), bone fractures, craniosynostosis, Coffin-Lowry syndrome, fibrodysplasia ossificans progressive, fibrous dysplasia, Fong Disease (or nail-patella syndrome), giant cell tumor of bone, greenstick fracture, hypophosphatasia, Klippel-Feil syndrome, metabolic bone disease, nail-patella syndrome, osteoarthritis, osteitis deformans (or Paget's disease of bone), osteitis fibrosa cystica (or osteitis fibrosa, or Von Recklinghausen's disease of bone), osteitis pubis, condensing osteitis (or osteitis condensas), osteochondritis dissecans, osteochondroma (bone tumor), osteogenesis imperfecta, osteomalacia, osteomyelitis, osteopenia, osteopetrosis, osteoporosis, osteosarcoma, porotic hyperostosis, primary hyperparathyroidism, renal osteodystrophy, Salter-Harris fractures, and water on the knee.

### Imaging Agents

Nanogels conjugated to one or more imaging agents are used to detect sites of localized activity targeted by one or more ligands of the nanogel. In some embodiments, the nanogel comprises one or more targeting agents and further comprises one or more imaging agents, selected from the group comprising radiolabels, radionuclides, radioisotopes, fluorophores, fluorochromes, dyes, metal lanthanides, paramagnetic metal ions, superparamagnetic metal oxides, ultrasound reporters, x-ray reporters, and fluorescent proteins.

In some embodiments, radiolabels comprise ^{99m}Tc, ¹¹¹In, ⁶⁴Cu, ⁶⁷Ga, ¹⁸⁶Re, ¹⁸⁸Re, ¹⁵³Sm, ¹⁷⁷Lu, ⁶⁷Cu, ¹²³I, ¹²⁴I, ¹²⁵I, ¹¹C, ¹3N, ¹⁵O, ¹⁸F, ¹⁸⁶Re, ¹⁸⁸Re, ¹⁵³Sm, ¹⁶⁶Ho, ¹⁷⁷Lu, ¹⁴⁹Pm, ⁹⁰Y, ²¹²Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ⁶⁷Cu, ¹⁰⁵Rh, ¹¹¹Ag, and ¹⁹²Ir. In some embodiments, paramagnetic metal ions comprise Gd(III), Dy(III), Fe(III), and Mn(II). In some embodiments, ultrasound reporters comprise gas-filled bubbles such as Levovist, Albunex, or Echovist, or particles or metal chelates where the metal ions have atomic numbers 21-29, 42, 44 or 57-83. In some embodiments, x-ray reporters comprise iodinated organic molecules or chelates of heavy metal ions of atomic numbers 57 to 83.

In some embodiments, fluorophores comprise fluorochromes, fluorochrome quencher molecules, any organic or inorganic dyes, metal chelates, or any fluorescent enzyme substrates, including protease activatable enzyme substrates. In some embodiments, fluorophores comprise fluorescent silicon nanoparticles. Fluorochromes comprise far red, and near infrared fluorochromes (NIRF). Fluorochromes include but are not limited to a carbocyanine and indocyanine fluorochromes. In some embodiments, imaging agents comprise commercially available fluorochromes including, but not limited to Cy5.5, Cy5 and Cy7 (GE Healthcare); AlexaFlour660, AlexaFlour680, AlexaFluor750, and AlexaFluor790 (Invitrogen); VivoTag680, VivoTag-S680, and VivoTag-S750 (VisEn Medical); Dy677, Dy682, Dy752 and Dy780 (Dyomics); DyLight547, DyLight647 (Pierce); HiLyte Fluor 647, HiLyte Fluor 680, and HiLyte Fluor 750 (AnaSpec); IRDye 800CW, IRDye 800RS, and IRDye 700DX (Li-Cor); and ADS780WS, ADS830WS, and ADS832WS (American Dye Source) and Kodak X-SIGHT 650, Kodak X-SIGHT 691, Kodak X-SIGHT 751 (Carestream Health).

### Characterization of Nanogels

In some embodiments, nanogels are characterized by techniques comprising nuclear magnetic resonance (NMR), dynamic light scattering (DLS), transmission electron microscopy (TEM), and atomic force microscopy (AFM). In some embodiments, the chemical structures of polymers and nanogels are confirmed using NMR. In some embodiments, particle sizes of nanogels are measured using dynamic light scattering (DLS). In some embodiments, particle sizes of nanogels are measured using transmission electron microscopy (TEM). IN some embodiments, formation of crosslinked particles in nanogels are confirmed using atomic force microscopy (AFM).

Herein is introduced a facile method to produce nanogels using click chemistry (Kolb et al., Drug Discov. Today 2003) with free groups for surface modification which we employed to target several tissues, including bone. In certain experimental examples, the biopolymer dextran, modified separately with clickable alkyne or azide groups, was crosslinked within an inverse emulsion to result in nanoparticles with an excess of free unreacted groups for subsequent conjugation. Both free click-able groups were used to control the nanogel surface and internal properties. The nanogels, with an average diameter of 67 nm, were characterizable via NMR, underwent enzymatic degradation, exhibited negligible cytotoxicity, and demonstrated preferential uptake by macrophages in vitro. In vivo biodistribution studies found that dextran nanogels localized in lymph nodes, liver, spine and femur. Moreover, the bisphosphonate ligand reduced nanogel uptake in the liver by 43%. While non-targeted nanogels entered the bone marrow and were engulfed by F4/80-positive cells in this tissue, bisphosphonate-functionalized nanogels exhibited reduced F4/80-positive cell uptake and demonstrated binding to both cortical and trabecular bone lining the marrow cavities. Although the overall uptake into the F4/80-positive cells decreased, a secondary beneficial effect was noted by F4/80-positive cell depletion, suggesting an anti-osteoporotic capacity of the targeted nanogels.

### EXPERIMENTAL EXAMPLES

### Materials

11-azido-3,6, 9 trioxaundecan-1-amine (CAS 134179-38-7), 1,1′ carbonyldiimidazole (CAS 530-62-1) acetonitrile (CAS 75-05-8), anhydrous dimethyl sulfoxide (CAS 67-68-5), dextran from Leuconostoc mesenteroides (avg. 9,000-11,000 g/mol, CAS 9004-54-0), anhydrous dichloromethane (CAS 75-09-2), 4 pentyn-1-ol (CAS 5390-04-5), 4-(dimethylamino) pyridine (CAS 1122-58-3), 4-amino-1-hydroxy-1-phosphonobutyl phosphonic acid, monosodium (Alendronate sodium trihydrate) (CAS 121268-17-5), Tris(hydroxymethyl)aminomethane (CAS 77-86-1), Span™ 80 (Sorbitan monooleate) (CAS 1338-43-8), Sodium ascorbate, copper(II) sulfate, and solvents were purchased from Sigma Aldrich. 3-(2-{2-[2-(2-Azido-ethoxy)-ethoxy]-ethoxy}Azide-ethoxy)-propionic acid 2,5-dioxo-pyrrolidin-1-yl ester (Azide-PEG4-NHS ester) was purchased from Click Chemistry Tools. 6-Azidosulfonylhexyl-triethoxysilane (CAS 96550-26-4) was purchased from Gelest. Alexa Fluor® 647 alkyne, triethylammonium salt was purchased from Invitrogen. Double deionized water was obtained from a 18.2 MS2 Barnstead Nanopurifaction system.

### Experimental Methods—Preparation of Nanogels

Nanogels were synthesized in an inverse miniemulsion created using 573 mg of Span™ 80 dissolved in 15 mL cyclohexane in a glass vial with a magnetic stir bar. The aqueous phase consisted of 0.043 mg/mL of alkyne-dextran polymer, 0.014 mg/mL azide-dextran polymer, 40 mM sodium ascorbate, and 13 mM of copper(II) sulfate dissolved in water. The aqueous phase was mixed immediately, after the addition of solutes, with the oil phase and ultrasonicated in a water bath for 30-60 seconds. The reaction mixture was stirred at 350 rpm for 12-20 hours. All quantities were carefully determined to produce the miniemulsion and to result in nanogels. Careful precision in the particle synthesis method is required to avoid non-nanogel end products such as microparticles, no particles, or unwanted residue on the bottom of the beaker.

The nanogels were purified by centrifuging the miniemulsion at 16,000 rcf for 30 minutes before removing the supernatant. The pellet was resuspended in THF and centrifuged again and the supernatant was removed. The pellet was then suspended in water and dialyzed extensively using a 100,000 MWCO membrane for four days. Particles were lyophilized and stored at −20° C. Additional methods for nanogel preparation include the following:

### Alkyne-Dextran Synthesis

0.156 g of 1,1′ carbonyldiimidazole (CDI) (0.4817 mmole) was dissolved in 10 mL of dry DMSO under argon. 0.0898 ml of 4-pentyn-1-ol (0.4817 mmole) was then added drop-wise. After mixing for 2 hours under argon, the solution was added to 500 mg of dry dextran and 80 mg of dry 4-(dimethylamino) pyridine (DMAP), then stirred for another 48 hours. The polymer was purified by dialysis for 48 hours against deionized water.

### Azide-Dextran Synthesis

Under an inert atmosphere, 10 g of CDI (30.88 mmol) was dissolved in 120 mL of anhydrous DCM. 0.98 mL of 11-azido-3,6,9 trioxaundecan-1-amine (4.93 mmol) was then added drop-wise to the solution. The reaction was stirred under argon for two hours and then quenched with 60 mL of a 1:1 acetonitrile: water mixture. After 5 minutes, the reacted material was evaporated under vacuum at 45 degrees Celsius to a solid white material. 1.00 g of dextran (approx. 0.10 mmol) was dissolved in 15 mL of DMSO under argon and then added to the activated azido powder. 160 mg of the DMAP (3.30 mmol) dissolved in 2 mL of anhydrous DMSO was added and then all the materials were stirred at room temperature for 48 hours under argon. The polymer was purified by dialysis for 48 hours against deionized water.

### Dextran Nanoparticle Radiolabeling Procedure

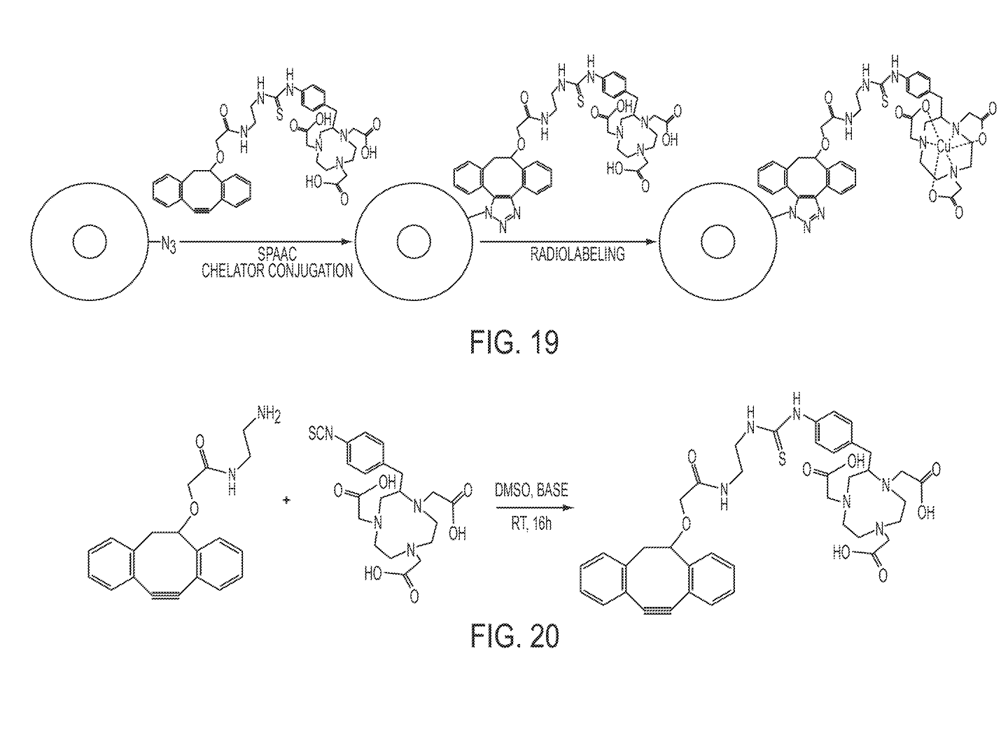

In one example, the procedure for radiolabeling the dextran nanoparticles includes two steps: the conjugation of the chelator (NOTA) to the nanoparticle and the chelation of the radiometal (64Cu) to the chelator-modified particle (FIG. 19).

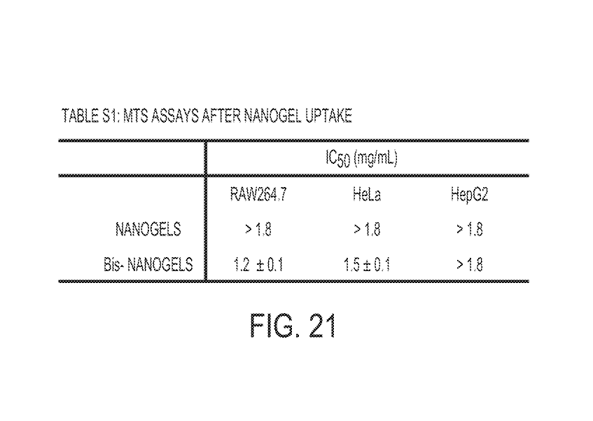

In the first step, catalyst-free, strain-promoted chemistry is used to conjugate a NOTA-modified dibenzocyclooctyne (DBCO-NOTA) to the azides decorating the outside of the nanoparticle. To this end, the particles are incubated with an excess of DBCO-NOTA in phosphate-buffered saline for 12 h at room temperature. This NOTA-modified dibenzocyclooctyne is synthesized via the facile thiourea bond formation reaction between a commercially available amine-modified dibenzocyclooctyne (Thermo-Fisher, Inc.) and a commercially available benzylisothiocyanate-modified NOTA (Macrocyclics, Inc.) (FIG. 20). In order to remove the excess DBCO-NOTA from the reaction solution, the particles are then dialyzed in phosphate-buffered saline for 48 h at 4° C. using a dialysis cartridge with a 100,000 molecular weight cut-off (Thermo-Fisher, Inc.).

In the radiolabeling step, the NOTA-modified particles are incubated with 64CuCl2 in 100 mM NH4OAc for 30 min at room temperature. The progress of the radiolabeling reaction is monitored using silica strip thin layer chromatography, an eluent of 50 mM EDTA pH 5.5, and a radiodetector. Once the radiolabeling reaction is complete, the newly labeled 64Cu-NOTA-particles are purified via size exclusion chromatography (e.g. GE Healthcare PD-10 column).

### Example 1

### Preparation and Characterization of Dextran Nanogel Particles

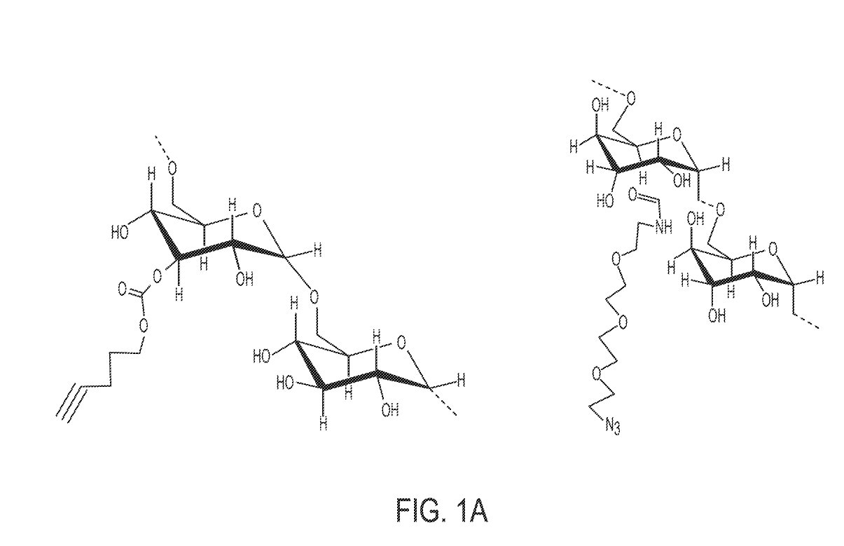

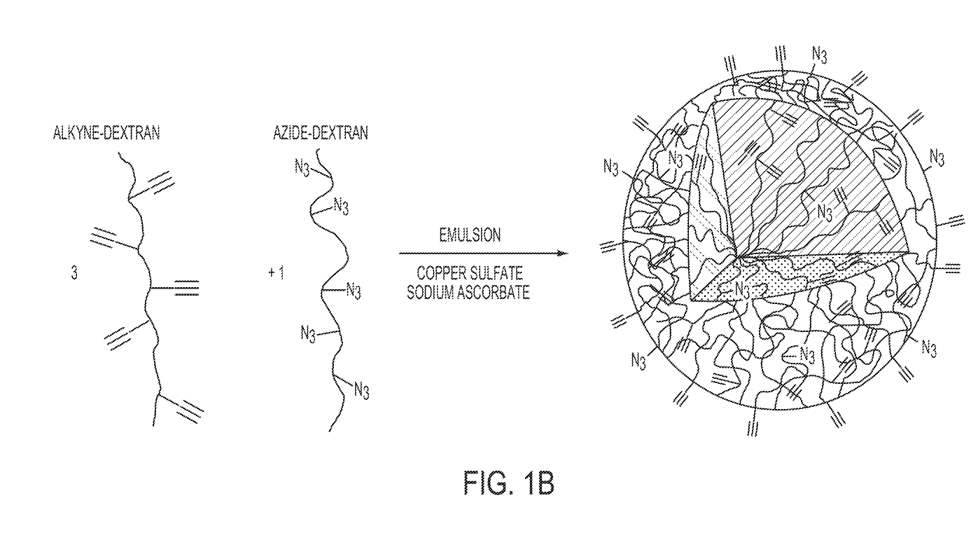

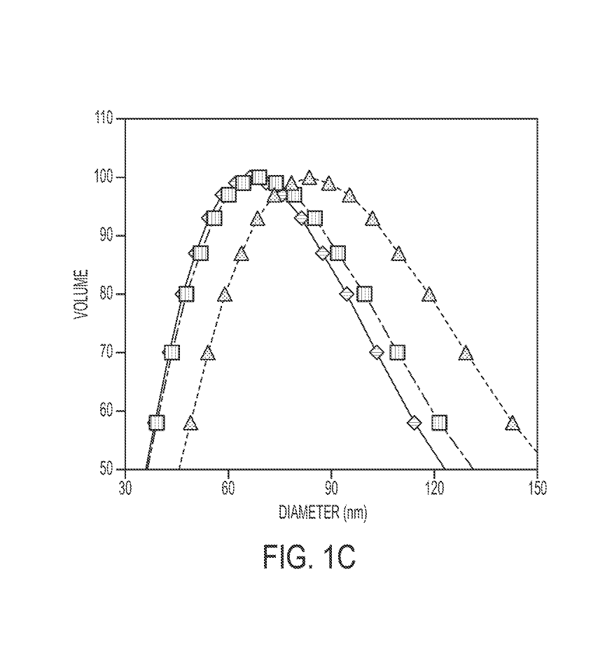

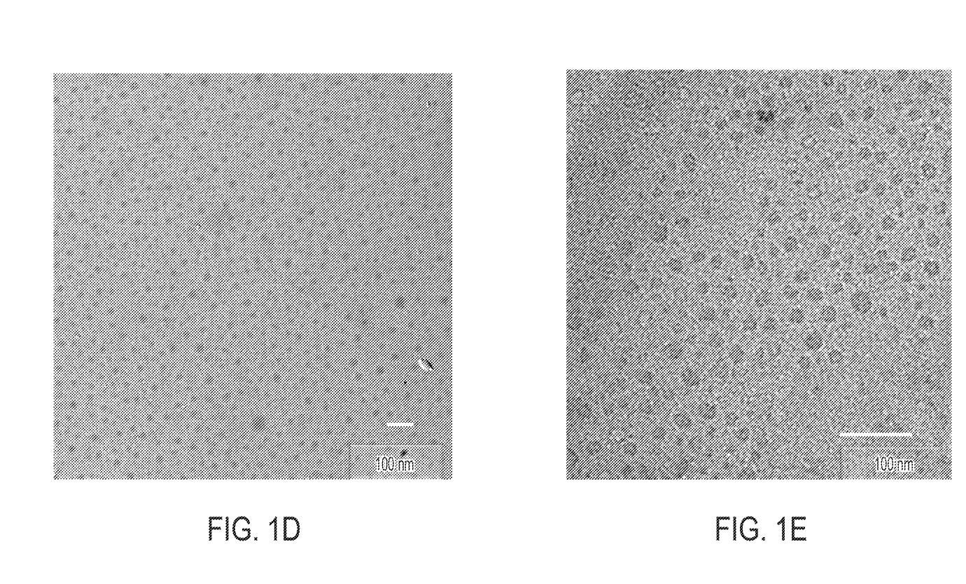

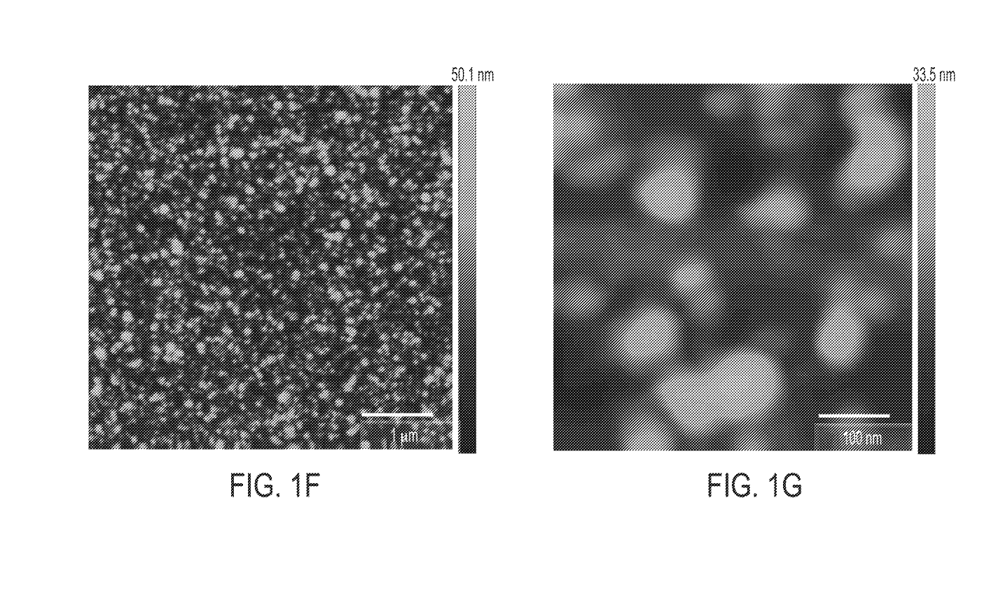

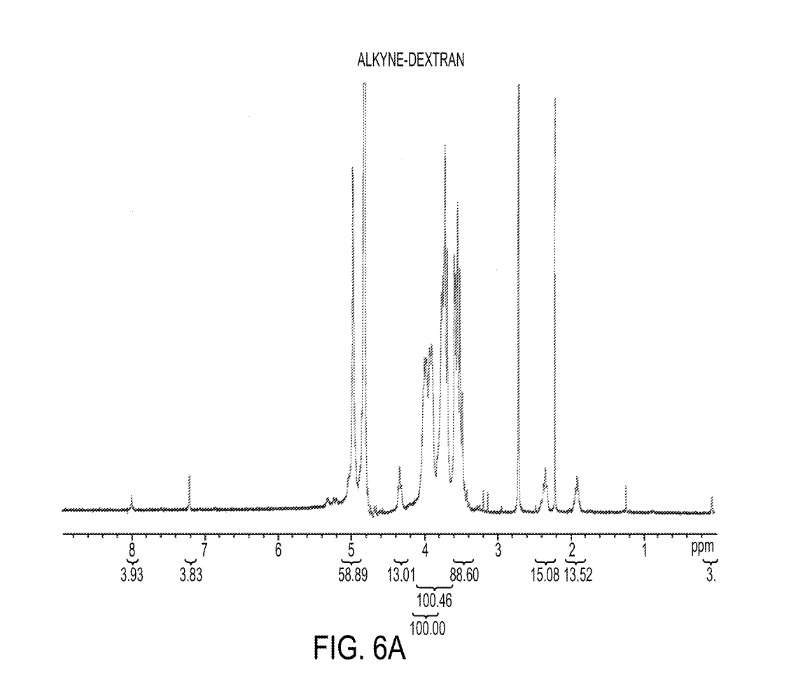

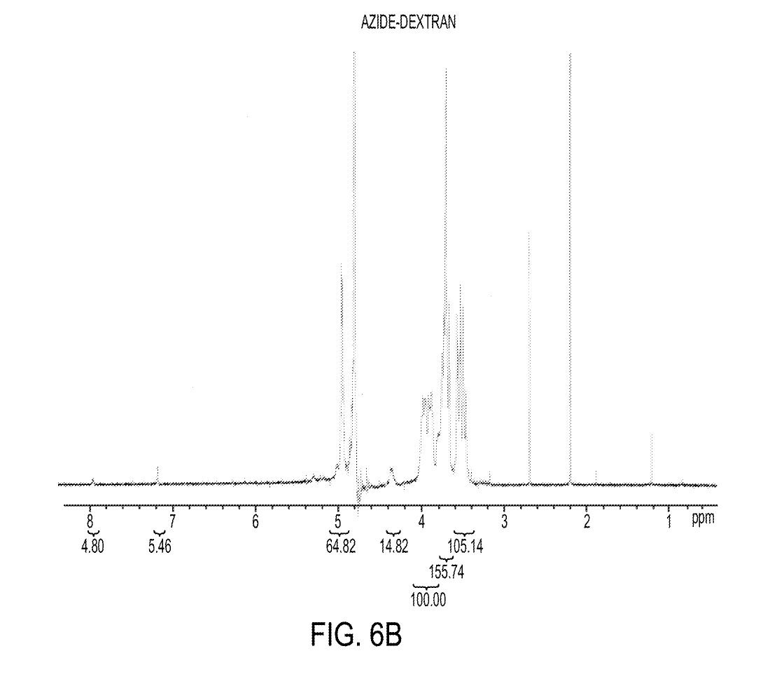

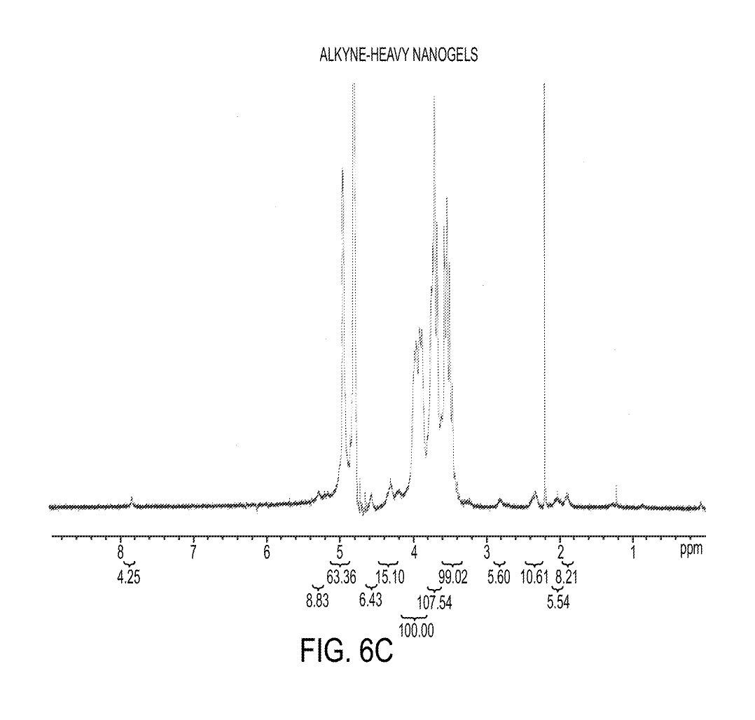

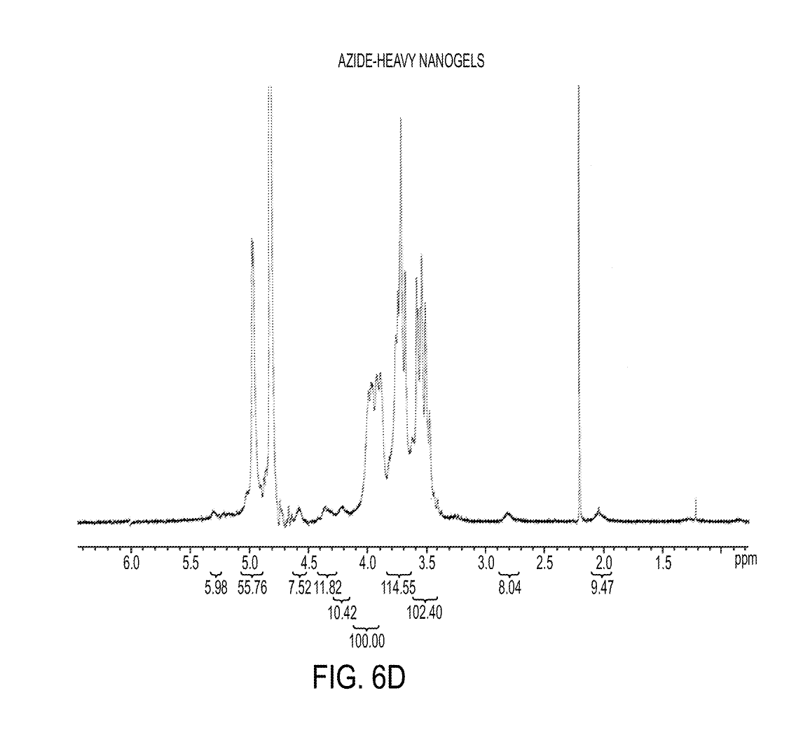

Nanogels composed of dextran were synthesized by initiating click chemistry within an inverse emulsion and characterized by several methods. Dextran polysaccharide (MW=10,000 Da) was modified via conjugation separately with a ligand bearing an alkyne group or azido group (FIG. 1A). The alkyne-functionalized dextran (alkyne-dextran), characterized via NMR (FIG. 6A and Supporting Experimental Methods), was synthesized with an alkyne ligand substitution ratio of 11.7% per glucose subunit, while azide-dextran exhibited a 4.6% substitution ratio (calculations described earlier).

### Nuclear Magnetic Resonance (NMR) of Dextran Polymers

### Native Dextran

¹H-NMR (500 MHz, D₂O-d6, δ/ppm): 4.0-5.0 (CH), 3.85-4.05 (CH), 3.6-3.7 (OH), 3.6, 3.8 (CH₂)

### Alkyne-Dextran

¹H-NMR (500 MHz, D₂O-d6, δ/ppm): Native dextran: 4.0-5.0 (CH), 3.85-4.05 (CH), 3.6-3.7 (OH), 3.6, 3.8 (CH₂). Alkyne-dextran additional peaks: 1.85, 2.32, 4.32 (CH₂), 2.695 (CH). The degree of substitution of alkyne-dextran was calculated from the ¹H-NMR spectrum by spectral integral ratios of the alkyne ligand protons relative to native dextran at 1.9, 2.1, and 4.15 ppm (CH₂) to the dextran proton at 4.9 ppm (CH): The alkyne ligand signal (15.08+13.57+13.01)=41.6. There are 6 protons on the alkyne chain, therefore the normalized added value is ˜41.6/6=6.9, and 6.9/58.89=11.7%. Thus, 11.7% of the glucose subunits are substituted with an alkyne ligand.

### Azide-Dextran

¹H-NMR (500 MHz, D₂O-d6, δ/ppm): Native dextran: 4.0-5.0 (CH), 3.85-4.05 (CH), 3.6-3.7 (OH), 3.6, 3.8 (CH₂). Azide-dextran additional peaks: 3.04, 3.4, 3.54, (CH₂), 8.00 (NH). The degree of substitution of azide-dextran was calculated from the ¹H-NMR spectrum by spectral integral ratios of the added azido ligand protons relative to native dextran peaks at 3.04, 3.4, and 3.54 ppm (CH₂) to the dextran proton at 4.9 ppm (CH): The azido ligand signal is (100+155+107)−(100+11+102)=48.7. The azido ligand contains 16 protons, therefore the normalized added value is ˜48.7/16=3, and 3/64.8=4.6%. Thus, 4.6% of glucose subunits are substituted with an azido ligand.

### Nuclear Magnetic Resonance (NMR) Studies of Nanogels

Lyophilized nanogels were dispersed in deuerated water at a concentration of 10-15 mg/mL for ¹H-NMR.

### Alkyne-Heavy Nanogels

Native dextran: 4.0-5.0 (CH), 3.85-4.05 (CH), 3.6-3.7 (OH), 3.6, 3.8 (CH₂); nanogels: 1.85, 2.10, 2.32, 3.9, 4.32 (CH₂), 7.9 (CH). The relative number of free alkyne ligands on 3:1 alkyne-dextran:azide-dextran (alkyne-heavy) nanogels was calculated using the ¹H-NMR spectral integral ratios of the protons of the added alkyne ligand at 1.9, 2.1, (CH₂) to the dextran proton at 4.9 (CH). Alkyne ligand peak areas (10.61+8.21)=18.82. The alkyne chain harbors 4 protons, therefore the normalized area ˜18.82/4=4.7. 4.7/63.36=7.4%. Therefore, 7.4% of the glucose subunits are substituted with an alkyne ligand. This decrease relative to the 11.7% on alkyne-modified dextran polymer is due to the cycloaddition of alkyne groups as well as dilution with azide-dextran polymer in the nanogel. Azido group signal was too low to measure.

### Azide-Heavy Nanogels

¹H-NMR (500 MHz, D₂O-d6, δ/ppm): Native dextran: 4.0-5.0 (CH), 3.85-4.05 (CH), 3.6-3.7 (OH), 3.6, 3.8 (CH₂); nanogels: 1.85, 2.32, 4.32 (CH₂), 2.695 (CH). The quantities of free alkyne and azide ligands in 1:3 alkyne-dextran:azide-dextran (azide-heavy) nanogels was calculated by ¹H-NMR spectrum from spectral integral ratios of protons related to the added alkyne ligand at 1.9 ppm and 2.1 ppm (CH₂) relative to the dextran proton at 4.9 ppm (CH). No measurable alkyne peaks were present in the spectrum, therefore it can be concluded that very few free alkyne groups were present. Azido group quantities were also too low to measure.

The nanogel particles were assembled by clicking the two modified dextran polymers together in either a 3:1 or 1:3 alkyne-dextran:azide-dextran ratio within an inverse emulsion (Bencherif et al., Biomat. 2009), producing alkyne-heavy or azide-heavy particles respectively (FIG. 1B). The alkyne-azide cycloaddition reaction between substituted dextrans was initiated with Cu+2 and sodium ascorbate added to the aqueous phase before emulsification with cyclohexane and a lipophilic surfactant. The resulting nanogels were characterized by NMR upon dispersing in deuterated water after purification. The spectra (FIG. 6A-D) exhibit diminished alkyne peaks and allow quantification of the remaining excess alkyne groups. The alkyne-heavy particles contain a final alkyne ligand substitution ratio of 7.4% with respect to the total number of glucose subunits contained in the particle. The NMR spectra show negligible azido group signal within both alkyne-heavy and azide-heavy nanogels, possibly due to low intrinsic signal strength of the group or restricted ligand mobility due to preferential localization within the particle instead of on the surface (Nystrom et al., Polym. Sci. Pol. Chem. 2009).

### Dynamic Light Scattering and Transmission Electron Microscopy

Dynamic light scattering (DLS) measurements of nanogels dispersed in PBS exhibited mean diameters of 67 nm and 86 nm for alkyne-heavy and azide-heavy particles, respectively, suggesting relatively monodisperse particle sizes (FIG. 1C). Particle sizes were determined by dynamic light scattering measurements with a ZetaPALS (Brookhaven) light scattering apparatus using a 90° excitation/collection orientation.

Samples for TEM were prepared by spreading the solution onto a carbon film-coated grid. Images were obtained using a JEOL 200CX electron microscope operated at 150 kV. Transmission electron micrographs (TEM) of alkyne-heavy nanogels show particles of homogenous electron densities with sizes between 20 and 40 nm (FIG. 1D-E). These differences between size measurements in aqueous medium (DLS) and dry (electron microscopy) are consistent with other nanogel types (Fisher et al., Pharm. Res. 2009).

### Atomic Force Microscopy

Nanogels were imaged by atomic force microscopy (AFM) to confirm formation of crosslinked particles. Silicon wafers were cleaned by sonicating in acetone for 10 minutes and methanol for 10 minutes. The wafers were then dipped in water, then isopropyl alcohol, then acetone, then isopropyl alcohol, and water. One drop of 6-Azidosulfonylhexyl-triethoxysilane was added to 10 mL of ethanol. Wafers were soaked for 30 seconds in this solution then transferred to a water bath. Wafers were dried by ultrapure nitrogen and dextran particles were reacted on this surface. Dextran particles were dissolved in water at a concentration of 25 mg/mL and mixed with the same amount of copper and ascorbate as used in the synthesis of the particles. Once the ascorbate is added to the dextran solution, one drop was placed on the wafers to react for 2 hours before rinsing with water and drying with ultrapure nitrogen. The wafers were then analyzed by AFM.

Silicon functionalized with an azido-silane compound (6-azidosulfonylhexyl-triethoxysilane) was used to promote alkyne-heavy nanogel attachment via cycloaddition conducted on the silicon surface. Height measurements, conducted in air, show evidence of surface-adsorbed spherical particles which confirm the sizes observed in the TEM micrographs (FIG. 1F-G).

### Example 2

### Nanogel Functionalization

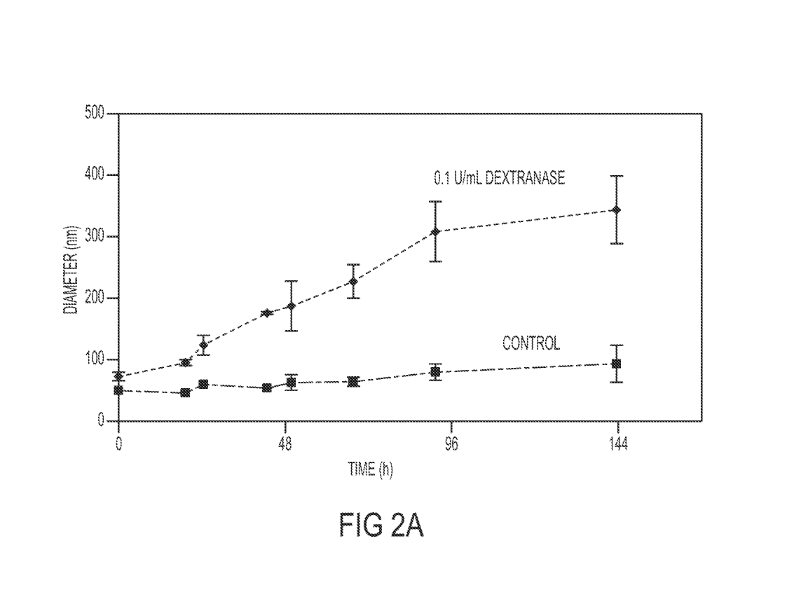

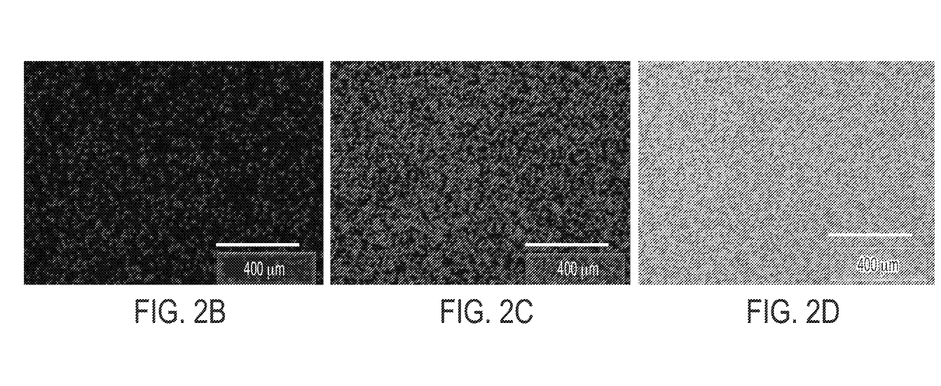

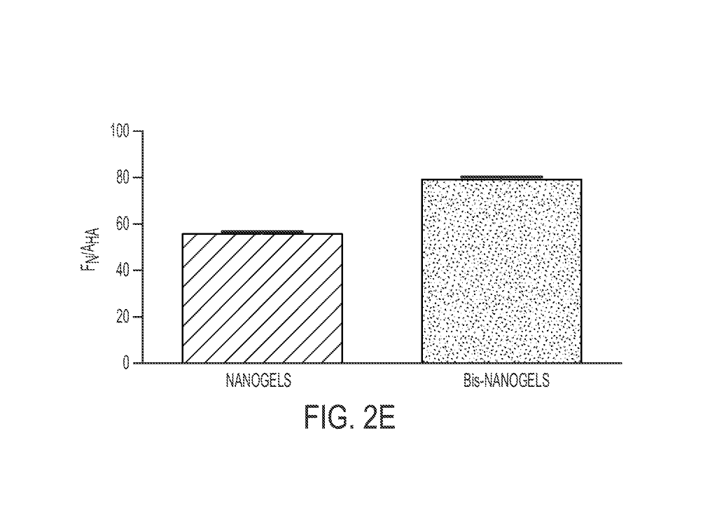

The nanogels showed evidence of enhanced degradation in the presence of dextranase. The nanogels, kept in pH 6.0 buffer to maximize dextranase efficiency, swelled to approximately 500% of their original size within 6 days of dextranase introduction (FIG. 2A). This behavior is consistent with other investigators' nanogel systems which demonstrate swelling as a result of the degradation of intra-particle crosslinks (Smith et al., Anal. Chem. 2010). Particles in dextranase-free buffer exhibited comparatively slight swelling behavior.

The astoichiometric excess of alkyne or azido groups allowed nanogel post-functionalization with two different moieties. Alkyne-heavy nanogels were functionalized with a bisphosphonate-presenting group containing a labile azido moiety, synthesized from alendronate precursor. The bisphosphonate-functionalized nanogels exhibited little change in size compared to unfunctionalized alkyne-heavy nanogels (FIG. 1C), with a peak diameter averaging 69 nm. The minority clickable group was also present in the nanogels and used for functionalization with a second ligand. The azido group in alkyne-heavy nanogels, although undetected by NMR spectroscopy, was functionalized with Alexa Fluor 647 containing an alkyne moiety and resulted in fluorescent nanogels post purification. The nanogels contained approximately 0.3 nmol of fluorophore per milligram of particles according to absorption spectrophotometry. Bi-functionalized nanogels exhibiting both fluorophore and bisphosphonate ligands contained 0.19 nmol of fluorophore per milligram of particle.

### Hydroxyapatite Binding Assay

To assess the binding capacity of functionalized nanoparticles to hydroxyapatite (HA), the wells of a cell culture plate were coated with HA and the nanoparticle suspension was added. The plate was left to incubate at room temperature overnight before washing. Fluorescence microscopy was used to evaluate the retention of nanoparticles over the HA layer. To prepare these HA layers, a 24 well plate was first treated with a solution of dopamine hydrochloride (2 mg/mL) in 10 mM Tris(hydroxymethyl)aminomethane under orbital shaking at room temperature for 24 hours. Then the wells were washed several times with deionized water and dried at 37° C. for additional 24 hours. Then, 0.5 mL of a previously prepared simulated body fluid (SBF) solution, an acellular solution with the same inorganic composition as human plasma (Kokubo et al., Biomat. 2006), was added to every well of the plate, which was then incubated at 37° C. under orbital shaking. The SBF solution was changed every day with fresh SBF for two weeks, and after this time HA deposition was confirmed through microscopy. Dextran nanoparticles were added to the wells and the plate was orbitally shaken overnight at 37° C. After this, the plates were rinsed with water several times, and then fluorescence microscopy was carried out to determine the presence of grafted nanoparticles to the HA layer.

A binding study demonstrates that bisphosphonate functionalization increases nanogel affinity to the bone mineral hydroxyapatite (FIG. 2B-D). Hydroxapatite, adhered to a polystyrene surface, was interrogated with either dextran nanogels or bisphosphonate-functionalized nanogels. Both nanogel constructs were conjugated to Alexa Fluor 647 fluorescent dye using their minor (azido) clickable group. After 12 hours of incubation, bisphosphonate-labeled nanogels exhibited significantly higher binding to hydroxyapatite, as demonstrated by a 23% higher emission intensity on the hydroxyapatite particles, relative to alkyne-heavy nanogels, quantified by normalizing mean fluorescence to the hydroxyapatite-covered area.

### Example 3

### Cellular Uptake and Cytotoxicity of Dextran Nanogels

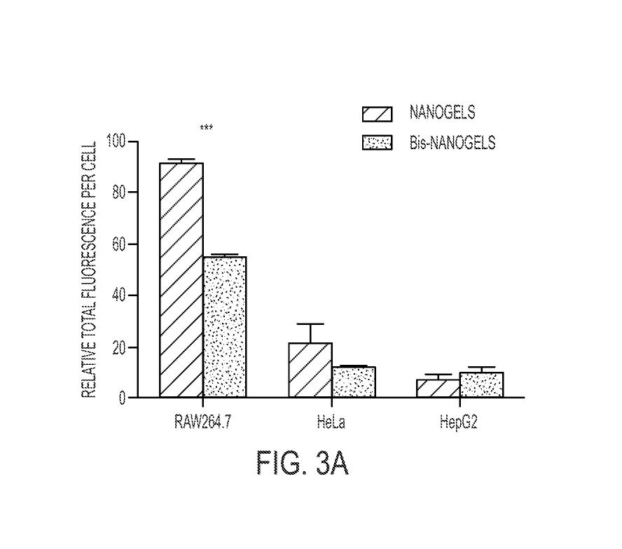

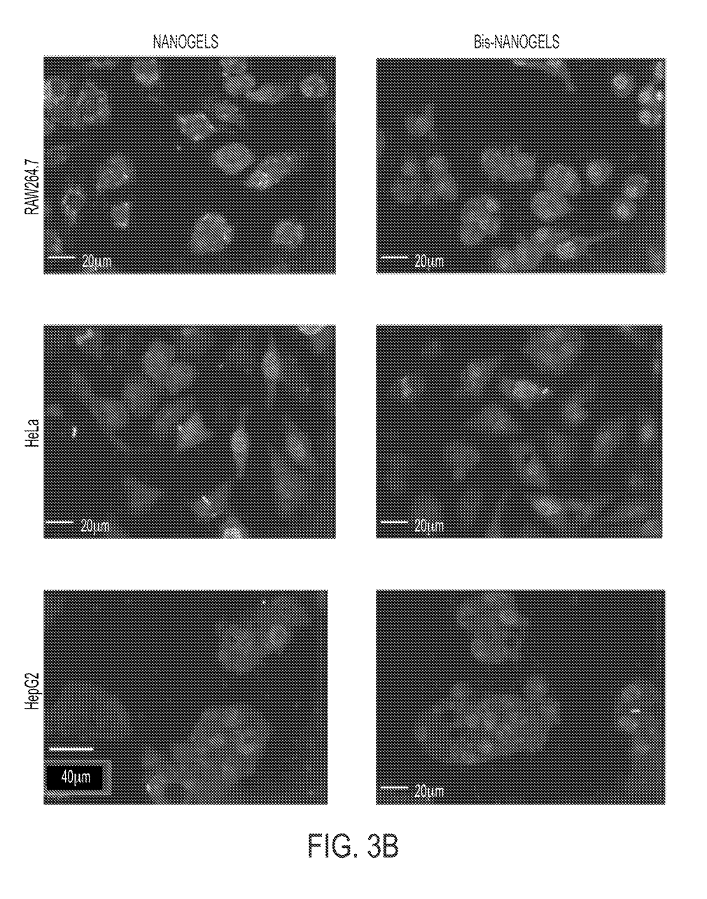

Dextran nanogels demonstrated higher uptake by macrophages than epithelial cells and hepatocytes in vitro, and they exhibited negligible cytotoxicity in all studied cell types. RAW264.7 cells (murine macrophage cell line) showed a 4-fold increase in uptake of Alexa Fluor 647-labeled nanogels as compared to HeLa or hepatocellular carcinoma (HepG2) cell lines (FIG. 3A).

### Confocal Microscopy and MTS Assay

To use confocal microscopy on adherent cells, RAW264.7 (20,000 cells/well), HeLa (16,000 cells/well) and HepG2 (20,000 cells/well) were seeded in black, clear bottom tissue-culture treated 96-well plates (Greiner) in 130 μl of growth medium (DMEM with 10% v/v FBS) and incubated in a humidified, 5% CO₂ atmosphere at 37° C. After 2 hrs, 20 μL aliquots of Alexa Fluor 647-labeled nanogel (non-functionalized or bisphosphonate-functionalized) solutions were added, and plates were incubated in a humidified, 5% CO₂ atmosphere at 37° C. for 24 hrs. The final nanogel concentration in each well was 200 μg/mL. Cells were rinsed with PBS, fixed with 3.7% formaldehyde (150 μL) for 10 min followed by quenching with BSA (150 μL of 10 mg/ml in PBS) and stored in PBS with Hoescht nuclear stain. Images were acquired using an Opera spinning disc confocal system (Perkin Elmer), and data was analyzed using Acapella Software (Perkin Elmer). Fluorescent intensity was normalized to amount of fluorophore functionalized in nanogels. Each condition was performed in triplicate.

In vitro measurements, collected in a 96-well plate format via high-throughput confocal microscopy, resulted in over 90 images of confluent cells under each condition. The images were processed in order to measure total corrected fluorescence intensity per cell (FIG. 3B). Bisphosphonate functionalization in nanogels attenuated cellular uptake by RAW264.7, as suggested by the 40% reduction in fluorescent intensity. Still, the fluorescent signal in these cells remained significantly higher than in HeLa and HepG2 cell lines.

Nanogel-mediated cytotoxicity was evaluated using the MTS assay. RAW264.7 (10,000 cells/well), HeLa (8,000 cells/well) and HepG2 (10,000 cells/well) were seeded in clear, tissue-culture treated 96-well plates (BD Falcon) in 100 μl of growth medium (DMEM with 10% v/v FBS) and incubated in a humidified, 5% CO₂ atmosphere at 37° C. After 24 hrs, the medium was replaced with 130 μL of fresh growth medium, and 20 μL aliquots of non-functionalized or bisphosphonate-functionalized nanogel solutions were added. The final nanogel concentration per well was varied from 0 L to 1.8 mg/mL. Plates were incubated in a humidified, 5% CO₂ atmosphere at 37° C. for 48 hrs. CeliTiter 96® AQueous One Solution Cell Proliferation Assay (MTS; Promega, Madison Wis.) was performed according to the manufacturer's instructions. Data was fitted to a sigmoidal curve, and the half maximal inhibitory concentrations (IC₅₀) were calculated as the polymer concentration corresponding to 50% cell survival. Each condition was performed in triplicate.

For non-functionalized nanogels, no measurable cell death was apparent in RAW264.7, HeLa and HepG2 cell lines even at concentration as high as 1.8 mg/mL (Table S1). Functionalization of bisphosphonate moieties in nanogels had a slight effect on cytotoxicity, particularly in Raw264.7 and HeLa cells, which showed IC50 values of 1.2 mg/mL and 1.5 mg/mL, respectively.

### Example 4

### Biodistribution of Nanogels

### Experimental Methods—Preparation of Fluorophore and Bisphosphonate Conjugation

Alkyne-heavy nanogels were dissolved in water to a concentration of 25 mg/mL. 0.5 mg Alexa Fluor 647 alkyne was dissolved in 50 uL of DMF and an aliquot of 10 uL was added to the nanogels. Sodium ascorbate was added to a final concentration of 40 mM and copper(II) sulfate was added to a final concentration of 13 mM. The sample was covered and reacted while vortexing for 12-20 hours. The samples were dialyzed against water in 12,000-14,000 MWCO membranes, covered, for 3-4 days with buffer changes twice daily before lyophilization.

To functionalize particles with bisphosphonate moieties, alendronate was conjugated to a heterobifunctional click chemistry reactant, azide-PEG4-NHS ester. An aliquot of 50 mg (0.154 mmol) of alendronate was dissolved in 30 mL of PBS and added to 0.077 mmol of Azide-PEG4-NHS ester dissolved in 7.71 mL of DMF. The reactants reacted at a 2:1 molar ratio to ensure excess alendronate. The reaction was magnetically stirred at room temperature overnight and solvent removed using a rotary evaporator.

Bisphosphonate-PEG4-Azide was dissolved in water to a concentration of 90 mg/mL. 22.5 mg of fluorophore-conjugated dextran nanogels were mixed with 400 uL of the bisphosphonate solution. Copper sulfate and sodium ascorbate were prepared and added in the same ratio as the fluorophore conjugation. The sample was reacted, covered and shaking, overnight and dialyzed using 12,000-14,000 MWCO membranes, covered, for 3-4 days with the water changed twice daily, followed by lyophilization,

### Animal Imaging

Animal experiments were performed according to local, state and federal regulations, and were approved by the Institutional Animal Care and Use Committee (IACUC) at Massachusetts Institute of Technology. Female 8-week old SKH-1 mice (Charles Rivers Laboratory) were intravenously (i.v.) injected via the tail vein with a single dose (100 μL of 15 mg/mL; 5 mL/kg) of Alexa Fluor 647-labeled nanogels (non-functionalized or bisphosphonate-functionalized) or PBS. At designated times after i.v. injection (1 hr, 4 hrs and 24 hrs), mice were anesthetized with isoflurane inhalation and whole-body image of mice was acquired using an IVIS spectrum imaging system (Xenogen). At 24 hrs, organs (heart, lungs, liver, spleen, kidneys, femur and spine) were harvested and imaged. Data was analyzed using Living Image® software. Background-subtracted fluorescence intensity was normalized to organ weight and amount of fluorophore bound to the nanogels as measured by absorbance spectroscopy.

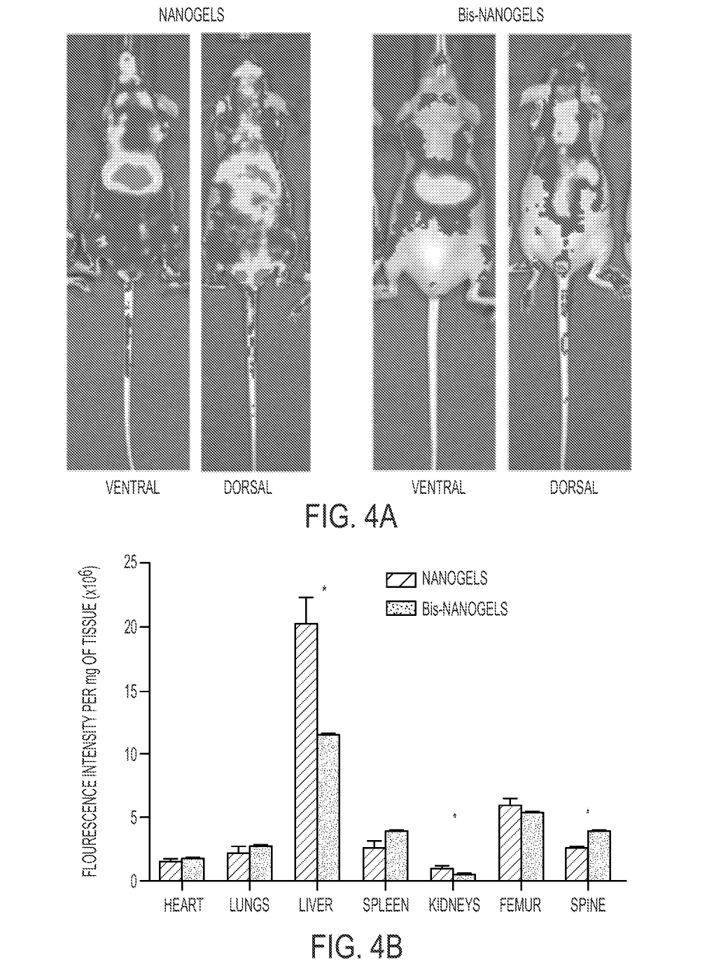

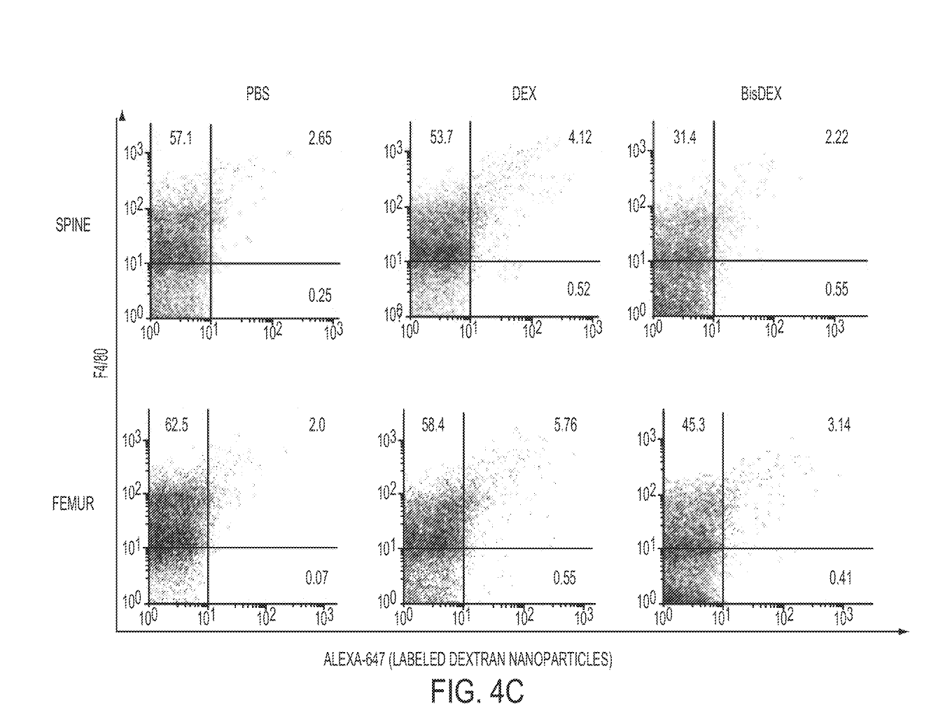

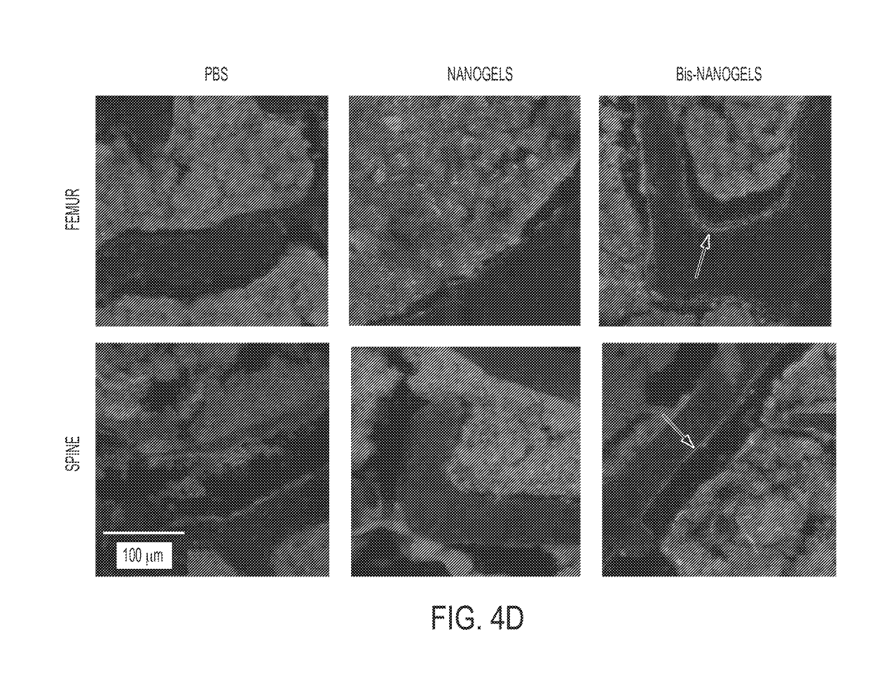

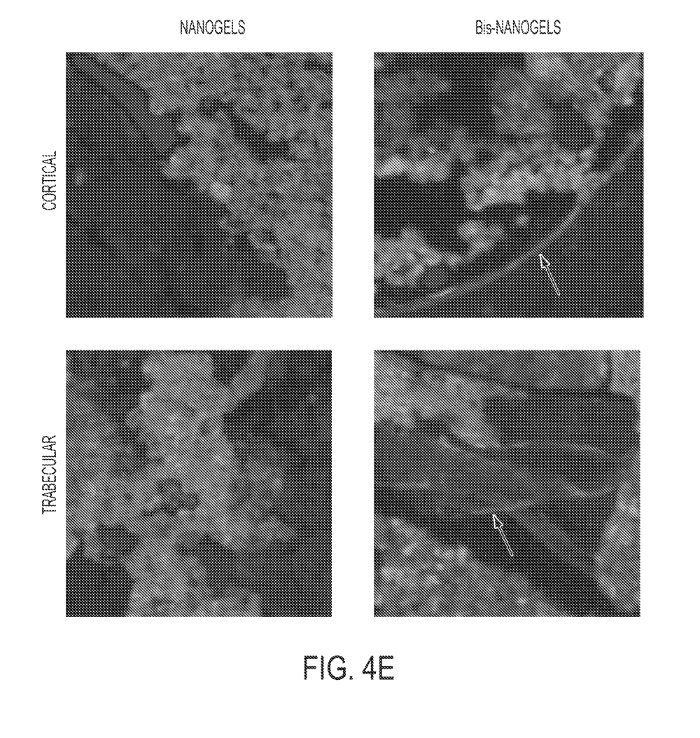

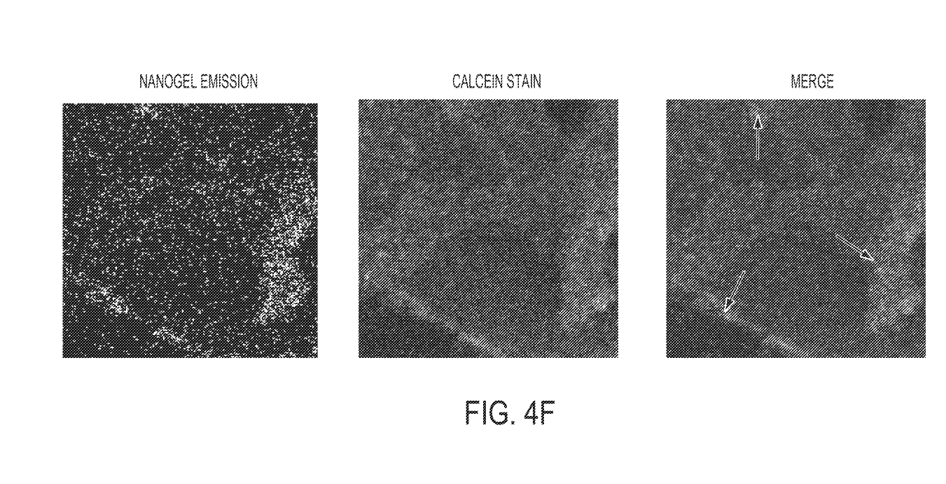

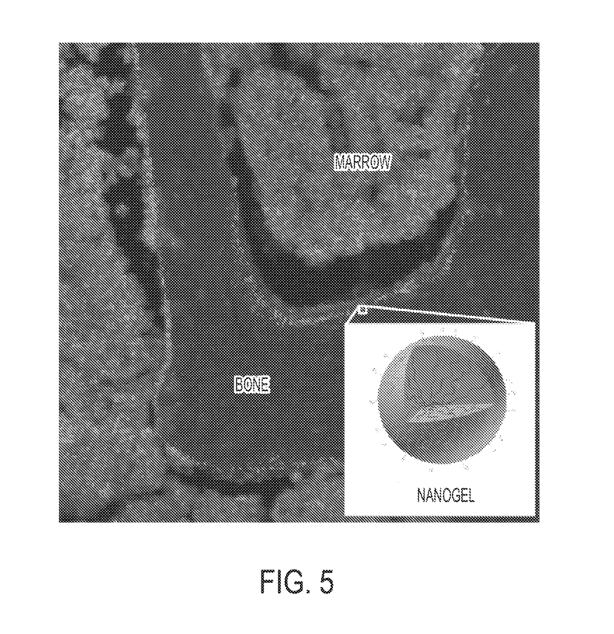

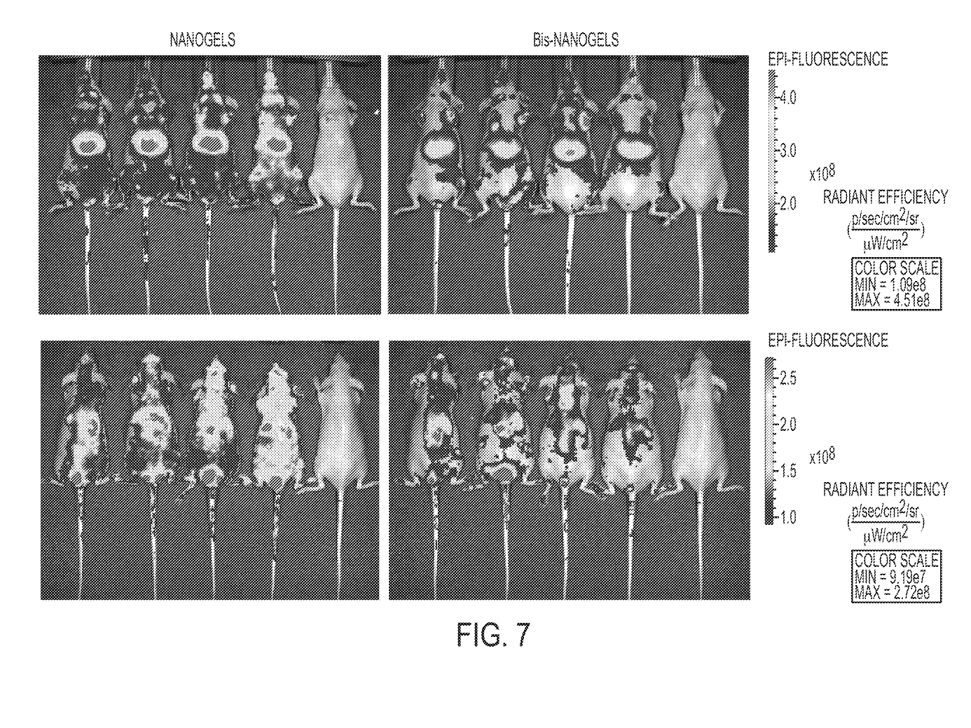

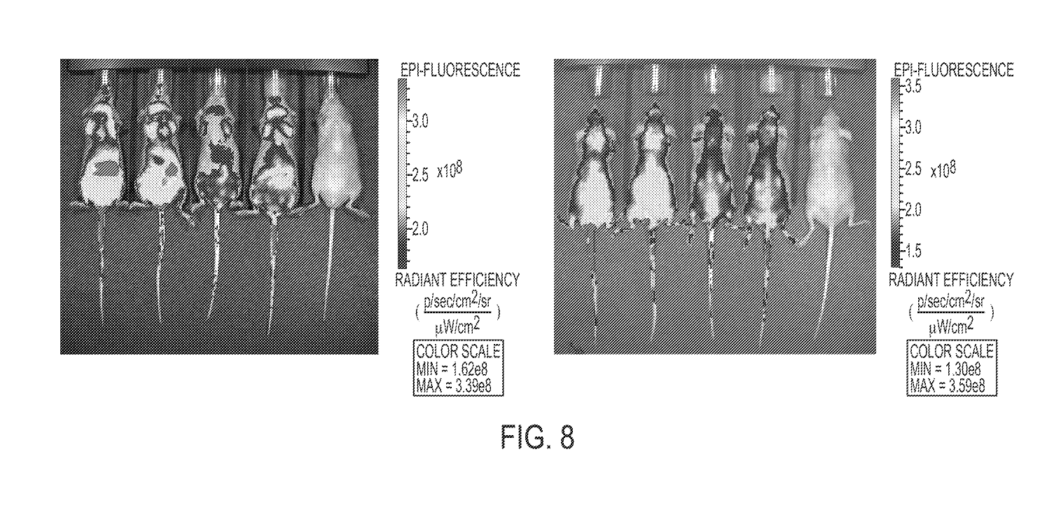

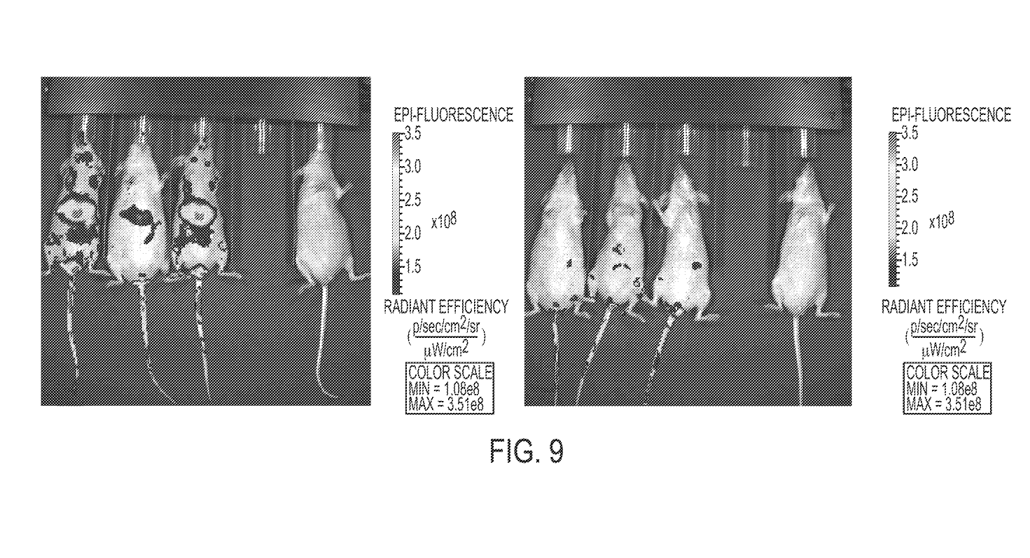

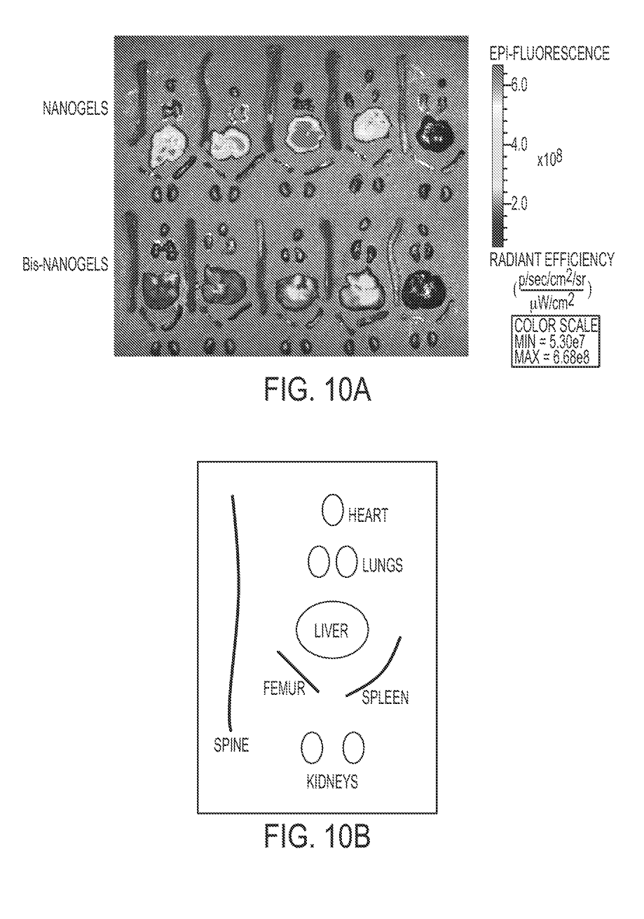

In vivo, bisphosphonate-functionalized nanogels exhibited spinal localization and attenuation of liver accumulation in murine biodistribution studies. Hairless SKH-1 mice were intravenously (i.v.) injected via the tail vein with a single dose of Alexa Fluor 647-labeled nanoparticles that were either un-functionalized or derivatized with a bisphosphonate ligand (100 μL; 75 mg/kg body weight). In vivo imaging of mice harboring alkyne-heavy nanogels showed generalized fluorescence in the body 24 hrs post-injection (FIG. 4A; All mice shown in FIG. 7). In the ventral view, a bright central fluorescent accumulation is apparent in the liver. In addition, pairs of fluorescent spots appear symmetrically at locations known to harbor cervical lymph nodes (Kobayashi et al., Acs Nano 2007). The dorsal image shows some generalized fluorescence throughout the body with localization in the body midsection and the centerline up to the head. For comparison, un-crosslinked dextran polymer demonstrates similar lymph node and centerline accumulation without liver localization (FIG. 8). Of note, five days post-injection of dextran nanogels, the in vivo whole-body fluorescence attenuates markedly (FIG. 9). Mice injected with bisphosphonate-functionalized nanoparticles exhibit attenuated fluorescence in the liver compared to non-functionalized dextran nanoparticles. Dorsally, the mice exhibit a higher relative localization of fluorescence up the centerline of the animal, especially at the midsection where the spine curves away from internal organs.

### Tissue Processing and Imaging

Spinal columns and femurs were collected from C57BL/6 mice 24 hr after i.v. delivery of either PBS, Dex, or BisDex nanoparticles, the last two of which are labeled with AlexaFluor-647. Tissues were excised, clean of excess soft tissues and placed in 4% paraformaldehyde overnight at 4° C. Following overnight fixation, all tissues were placed in a 10% EDTA, 1×PBS solution and kept at 4° C. The 10% EDTA solution was then replaced with fresh solution every 2 days for a total of 10 days after fixation. Samples were then embedded in optimal cutting temperature (OCT) compound (Cat #4583, Tissue-Tek, Sakura Finetek, Torrance, Calif.) and cryosections (10-20 μm) were prepared using a Leica CM1900 cryotome. Confocal images were then taken with a Zeiss 710 NLO confocal and AxioObserver Z1 microscope stand. Representative images of sample sections representing at least two regions in each spinal column or femur for each treatment group and time point are shown. Alexa-647-conjugated Dex or BisDex nanoparticles were excited at 633 nm with a HeNe laser (recorded emission range: 638-755) and are shown in red. Bone marrow and bone morphological features are shown in blue after being excited at 405 nm with a solid state laser (recorded emission range: 410-497 nm). Calcein staining was conducted on decalcified samples by fixing in 1% paraformaldehyde, washing, and incubating in a solution containing 0.5% calcein and 0.2M NaOH for 15 minutes. Samples were then rinsed with water and cover-slipped with Invitrogen Prolong Gold reagent.

Imaging of the harvested organs confirms spinal accumulation, as well as liver and kidney attenuation, of bisphosphonate-functionalized nanoparticles compared to non-functionalized dextran nanoparticles (FIG. 4B, 10). Fluorescence quantification of whole organs by near-infrared imaging, shown to closely approximate other techniques (Vasquez et al., PLoS One 2011), was conducted 24 hrs after i.v. injection. Accumulation in femur for both types of nanoparticle is also appreciable and, notably, contrasts with whole-animal imaging data which shows little apparent accumulation. In mice treated with bisphosphonate-functionalized nanoparticles, corrected fluorescent intensity significantly decreases in liver and kidneys by approximately 43% for each organ. Localization increases slightly in the spleen, and significantly in the spine, by 36%. Overall, incorporation of the bisphosphonate moiety induces significant modulation in nanogel biodistribution, which are not attributable to changes in particle size or charge alone.

### Fluorescence-Activated Cell Sorting (FACS) Analysis

Single-cell suspensions of freshly excised bone marrow were isolated from excised mouse femurs and spinal vertebrae, which were cut open with surgical razor blades and washed out with phenol red-free alpha-MEM (Cat. #41061, Gibco, Grand Island, N.Y.). Bone marrow suspensions were passed through 70 μm filters (Cat. #22363548, Fisher Scientific, Pittsburgh, Pa.), and then subjected to red blood cell lysis with 5 ml of 1×RBC lysis buffer (Cat. #00-4333, eBioscience, San Diego, Calif., USA) for 5 min at room temperature. The reaction was terminated by the addition of 20 ml of sterile 1×PBS. The cells remaining were centrifuged at 300-400 g at 4° C. and resuspended in a minimal volume (˜50 μl) of eBioscience Staining Buffer for antibody incubation. All samples were then incubated in the dark for 25 min at 4° C. with a fluorescently tagged monoclonal antibody specific for the F4/80 antigen (1 μl (0.5 μg) per sample; F4/80-FITC, Clone BM8, Cat. #11-4801, eBioscience). Background samples were similarly stained with FITC-labeled Rat IgG (1 μl per sample, Cat. #11-4321, eBioscience). Samples were washed, filtered, resuspended and analyzed as described (Doloff et al., Cancer Res. 2012).

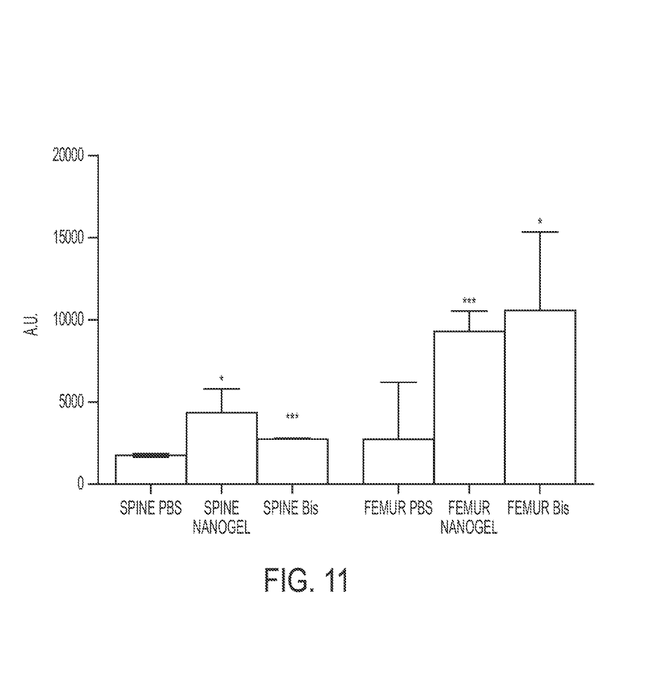

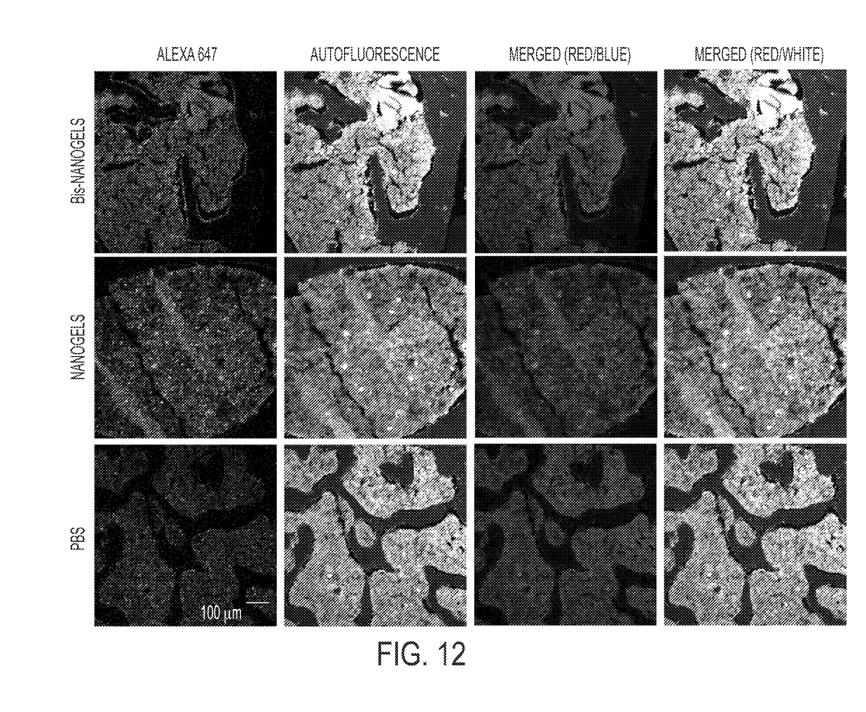

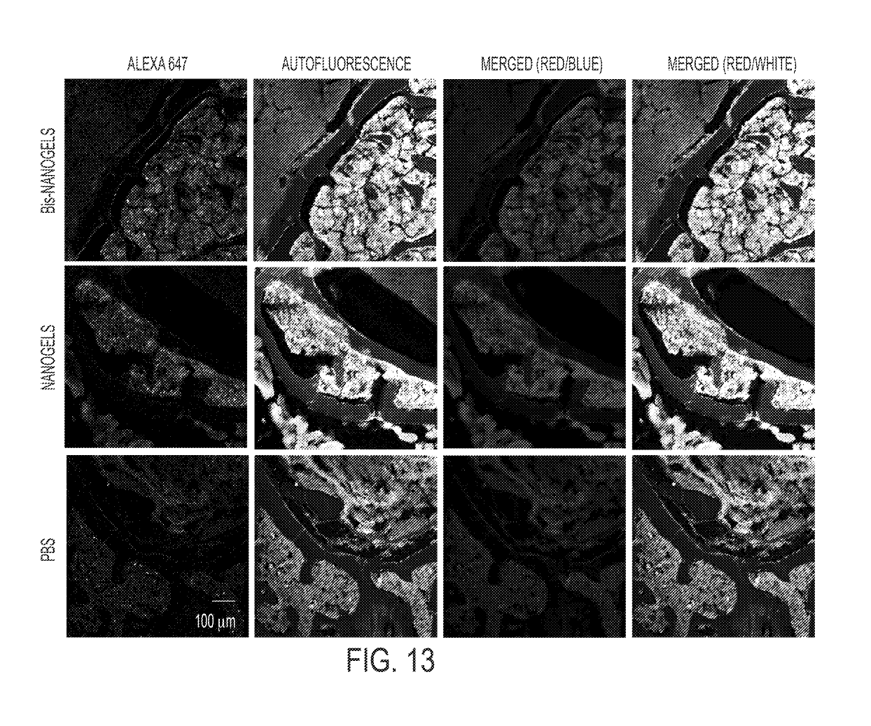

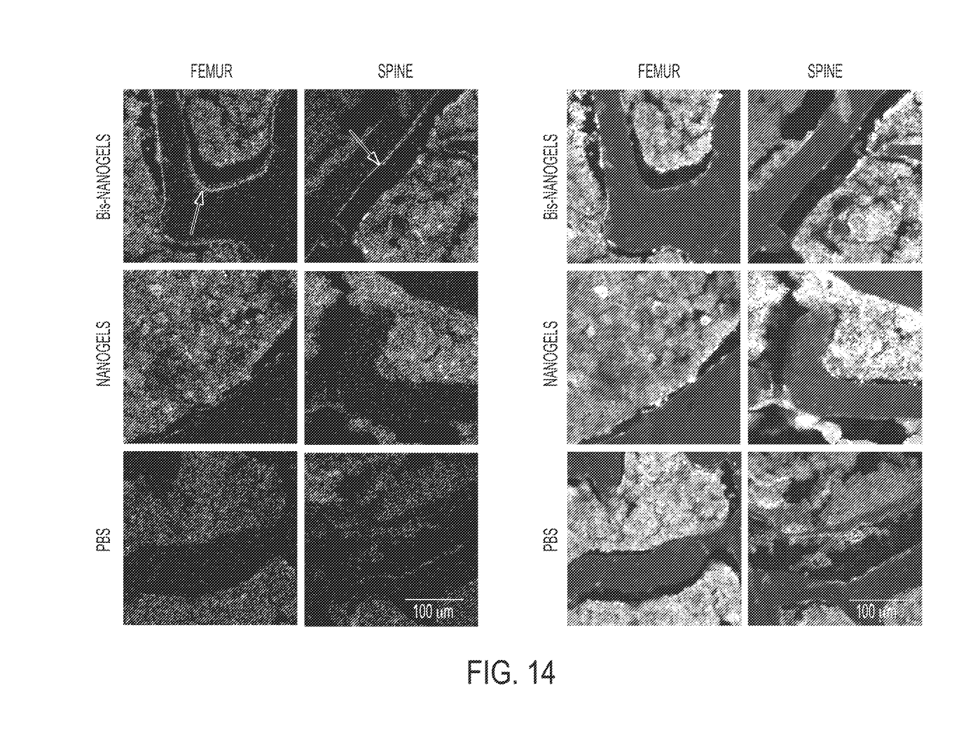

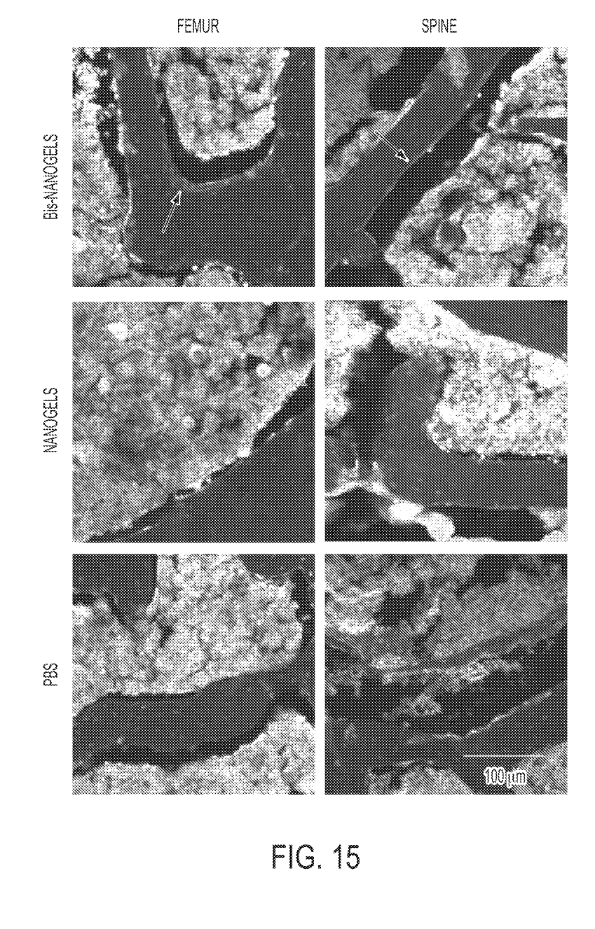

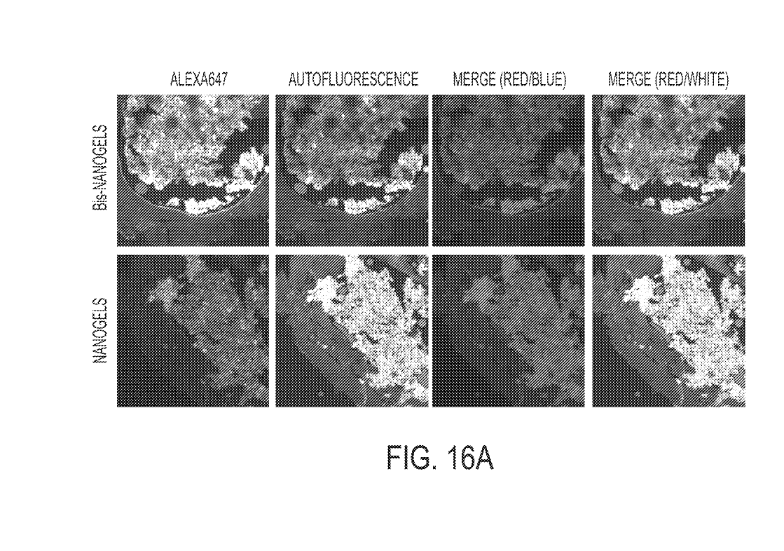

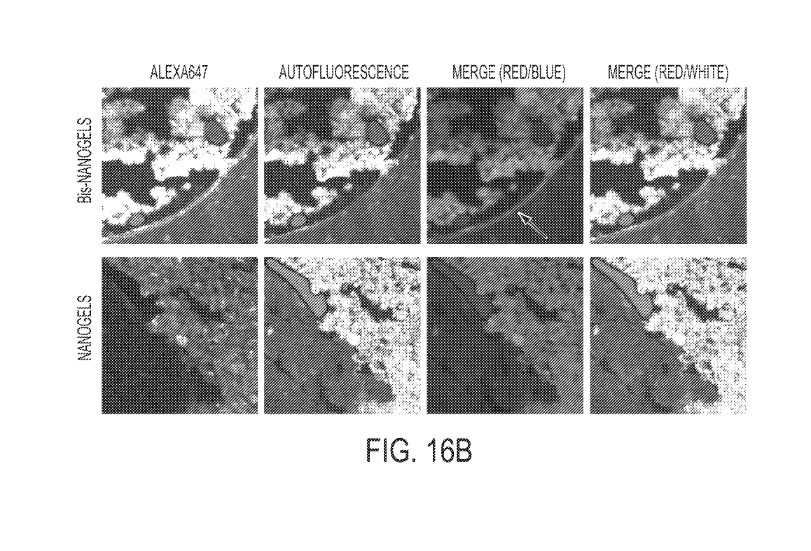

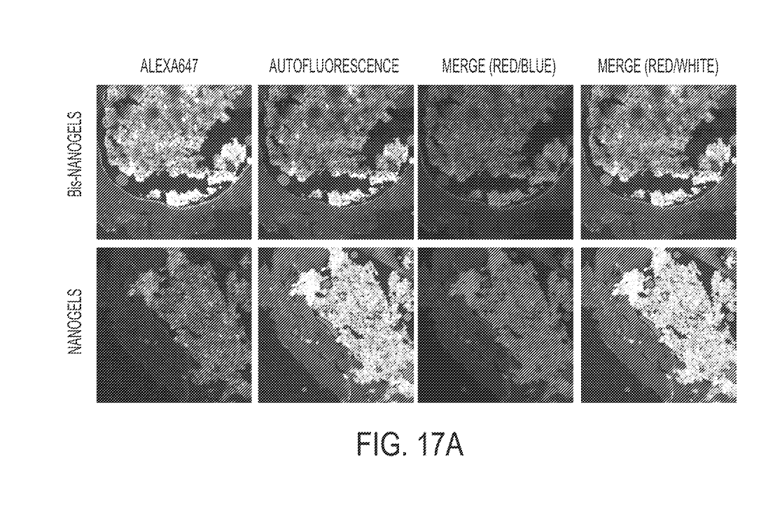

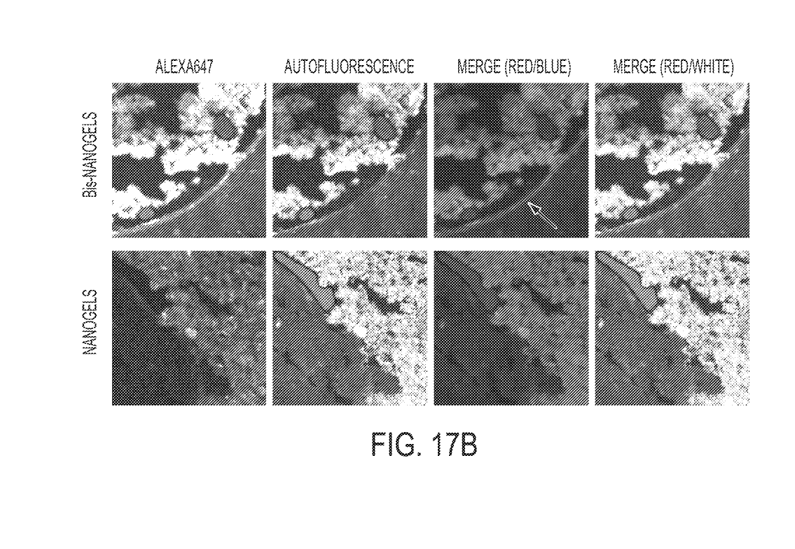

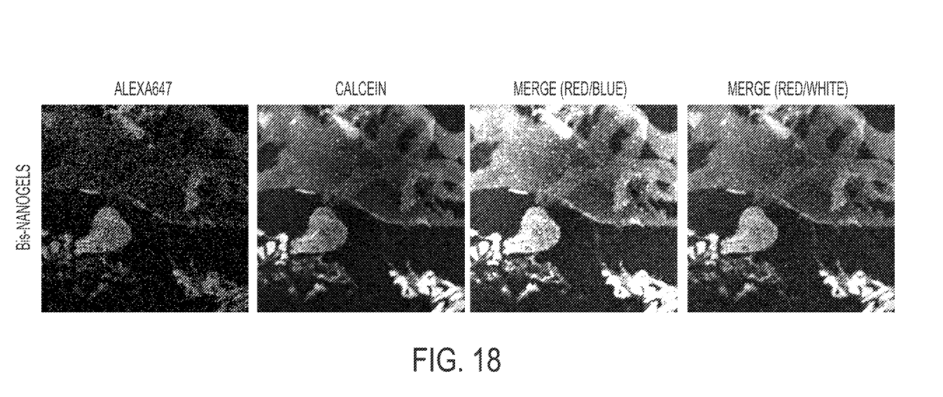

Dextran nanogels exhibited F4/80-positive cell uptake in femoral and spinal bone marrow, while bisphosphonate-functionalized nanogels attenuated this phenomenon, as shown by flow cytometry analysis. Bone marrow cells from femur and spine, harvested from Alexa Fluor 647-labeled dextran nanogel-treated mice, were labeled with FITC-conjugated F4/80 antigen-specific antibodies which target macrophages and osteoclast precursors (Lean et al., Bone 2000). For un-functionalized nanogels, flow cytometry measurements showed cells that are double positive for F4/80 and nanogels (4.12% cells for spine and 5.76% cells for femur), suggesting nanogel uptake by F4/80-positive cells (FIG. 4C). Bone marrow cells from Alexa 647-labeled, bisphosphonate-functionalized nanogel treated mice showed near-background levels of nanogel emission. Cells which were double positive for F4/80 and bis-nanogels were reduced to 2.22% cells for spine and 3.14% cells for femur. This treatment group also showed lower total levels of F4/80-positive cells, likely denoting a depletion of F4/80-positive cells relative to the control. Without wishing to be bound by any particular theory, depletion of macrophages and future osteoclasts, an effect of bisphosphonate, is thought to be the mechanism of its anti-osteoporotic effects (Fisher et al., Proc Natl Acad Sci USA 1999; Moreau et al., Biochem Pharmacol 2007; and Delmas et al., Curr Opin Rheumatol 2005). Furthermore, engulfment of nanogels by F4/80-positive cells was incomplete in the case of both dextran and bisphosphonate-modified nanogels, as many free nanogels were found within both the femoral and spinal marrow stroma, as detected by bulk fluorescence emission of rinsed cell supernatant (FIG. 11), signifying the presence of particles not accumulated in F4/80-positive cells.

Bisphosphonate-functionalized nanogels exhibited significant localization to the external HA in the marrow-bone interface in cryosectioned spinal and femoral tissue (FIG. 4D, 12-15). Un-targeted nanogels in both femur and spine distributed throughout the marrow without localizing to the marrow-bone interface. Within the femur, the localization of targeted nanogels at the interface was apparent in both cortical and trabecular bone (FIG. 4E, 16-17). The binding of bis-nanogels to newly-synthesized HA is suggested by the co-localization of bis-nanogels with the calcium ion-binding dye calcein in femur (FIG. 4F, 18).

The apparent unchanging femoral localization of targeted nanogels in FIG. 4B is likely caused by the large relative volume of marrow within the femur as well as the low surface area of the marrow-bone interface. Although the targeting ligand did not increase total nanogel signal in the femur, it did shift the femoral distribution in the bone from the marrow to the HA on the external parts of the cavity, resulting in greater bone localization of the targeted nanoparticles. This may be due both to the targeting ability of the functionalized particle as well as the F4/80-positive cell depletion effect of the ligand, resulting in less nanogel sequestration in phagocytes. Localization in spine exhibited an overall increase possibly due to the cancellous nature of the spinal vertebrae with a higher surface area-to-volume ratio of the marrow-bone interface in spine versus femur, allowing a larger percentage of the nanogels in the marrow cavities to bind to spinal versus femoral bone.

Modular, dextran-based nanogels were synthesized via a facile method to improve control over chemistry, characterization, and accumulation in frequent metastatic sites. The nanogels demonstrated degradability and displayed ligands for post-functionalization via click chemistry. The particles exhibited extremely low cytotoxicity in vitro, higher uptake by macrophages versus hepatocytes and epithelial cells, and were tolerated at high doses in vivo. Biodistribution studies showed significant localization in the liver and cervical lymph nodes, and bone marrow F4/80-positive cells uptake. Functionalization with a bisphosphonate ligand modulated this localization, reducing kidney and liver uptake by 43% and increasing accumulation in the spine by 36%. The targeting ligand resulted in significant nanogel localization at the HA-marrow interface in the walls of the marrow cavities in both femur and spine, and in both cortical and trabecular bone. Although the overall nanogel uptake into F4/80-positive cells was lower for the targeted nanogels, these nanogels depleted F4/80-positive cells within bone marrow, suggesting that the particles may contribute to a depletion of future ostoeclasts and might provide an anti-osteoporotic effect, which warrants further study.

The experiments demonstrate a facile technique to generate modular nanogels with controllable functionalization and targeting which hold potential for therapeutic applications towards bone disease.

### EQUIVALENTS

While the invention has been particularly shown and described with reference to specific preferred embodiments, it should be understood by those skilled in the art that various changes in form and detail may be made therein without departing from the spirit and scope of the invention as defined by the appended claims.

What is claimed is:

1. A nanogel for targeted tissue localization, the nanogel comprising residual functional groups of at least one type.

2. The nanogel of claim 1, wherein the nanogel comprises a targeting ligand.

3. The nanogel of claim 2, wherein the targeting ligand is a bisphosphonate for localization in bone.

4. The nanogel of claim 1, wherein the residual functional groups are of one or more types comprising one or more of the following: alkyne, azide, thiol (sulfydryl), alkene, acrylate, oxime, maliemide, NHS (N-hydroxysuccinimide), amine (primary amine, secondary amine, tertiary amine, and/or quarternary ammonium), phenyl, benzyl, hydroxyl, carbonyl, aldehyde, carbonate, carboxylate, carboxyl, ester, methoxy, hydroperoxy, peroxy, ether, meiacetal, meiketal, acetal, ketal, orthoester, orthocarbonate ester, amide, carboxyamide, imine (primary ketimine, secondary ketamine, primary aldimine, secondary aldimine), imide, azo (diimide), cyanate (cyanate or isocyanate), nitrate, nitrile, isonitrile, nitrite (nitrosooxy group), nitro, nitroso, pyridyl, sulfide, disulfide, sulfinyl, sulfonyl, sulfino, sulfo, thiocyanate, isothiocyanate, caronothioyl, thione, thial, phosphine, phosphono, phosphate, phosphodiester, borono, boronate, bornino, borinate, halo, fluoro, chloro, bromo, and/or iodo moieties.

5. The nanogel of claim 1, wherein the nanogel comprises a functionalized polymer.

6. The nanogel of claim 5, wherein the functionalized polymer is a polymer with at least 1%, 2%, 5%, or 7% of its monomer units having attached residual functional groups.

7. The nanogel of claim 6, wherein the monomer units comprise glucose subunits of a polysaccharide polymer.

8. The nanogel of claim 6, wherein the attached residual functional groups comprise alkyne groups.

9. The nanogel of claim 5, wherein the polymer is a polysaccharide.

10. The nanogel of claim 9, wherein the polysaccharide is dextran.

11. The nanogel of claim 1, wherein the nanogel has an average particle diameter between 5 nm and 1000 nm.

12. The nanogel of claim 11, wherein the average particle diameter is between 10 nm and 200 nm as measured via dynamic light scattering (DLS) of nanogel dispersed in PBS, or between 5 nm and 150 nm as measured via transmission electron micrograph (TEM).

13. The nanogel of claim 1, wherein the nanogel has a substantially monodisperse particle size.

14. The nanogel of claim 13, wherein the nanogel has a polydispersity index, Mw/Mn of less than 20.

15. A nanogel for targeted tissue localization, the nanogel comprising a polymer and one or more ligands coupled thereto and/or therewithin, the one or more ligands comprising: (i) one or more targeting agents, (ii) one or more therapeutic agents, and/or (iii) one or more imaging agents.

16. The nanogel of claim 15, wherein the one or more ligands are coupled to and/or within the nanogel by at least one of (i) physical entrapment, (ii) covalent conjugation, and (iii) controlled self-assembly.

17. The nanogel of claim 15, further comprising residual functional groups of at least one type.

18. The nanogel of claim 17, wherein the residual functional groups comprise alkyne moieties and/or azide moieties.

19. The nanogel of claim 15, the nanogel comprising one or more targeting agents comprising a bisphosphonate.

20. The nanogel of claim 19, wherein the one or more targeting agents comprise(s) a bisphosphonate for bone localization.

21. The nanogel of claim 15, the nanogel comprising one or more targeting agents and further comprising one or more therapeutic agents selected from the group consisting of estrogen, a radio-pharmaceutical, a corticoid, an anti-inflammatory agent, and a protein.

22. The nanogel of claim 15, the nanogel comprising one or more targeting agents and further comprising one or more imaging agents selected from the group consisting of radiolabels, radionuclides, radioisotopes, fluorophores, fluorochromes, dyes, metal lanthanides, and fluorescent proteins.

23. The nanogel of claim 15, wherein the one or more ligands comprises a peptide, polypeptide, and/or an antibody for binding cancer cell surface antigens/markers.

24. The nanogel of claim 1, wherein the one or more ligands comprises a ligand that is both a targeting agent and a therapeutic agent.

25. The nanogel of claim 24, wherein the therapeutic agent is a bisphosphonate.

26. The nanogel of claim 1, wherein the polymer is a polysaccharide.

27. The nanogel of claim 26, wherein the polysaccharide is dextran.

28. The nanogel of claim 1, wherein the nanogel has average particle diameter between 5 nm and 1000 nm.

29. The nanogel of claim 27, wherein the average particle diameter is between 10 nm and 200 nm as measured via dynamic light scattering (DLS) of nanogel dispersed in, or between 5 nm and 150 nm as measured via transmission electron micrograph (TEM).

30. The nanogel of claim 1, wherein the nanogel has substantially monodisperse particle size.

31. The nanogel of claim 30, wherein the nanogel has polydispersity index, Mw/Mn of less than 20.

32. The nanogel of claim 1, wherein the one or more ligands are conjugated to the polymer via alkyne (alkenyl) moieties and/or azide moieties.

33. The nanogel of claim 1, wherein the targeted tissue localization is localization in and/or on one or more members selected from the group consisting of bone marrow, liver, and lymph node.

34. The nanogel of claim 1, wherein the residual functional groups are free click-able functional groups.

35. The nanogel of claim 1, wherein the residual functional groups comprise unreacted groups for subsequent conjugation.

36.-66. (canceled)
